(19) United States
(12) Patent Application Publication
(10) Pub. No.: US 2025/0129409 A1
(43) Pub. Date: Apr. 24, 2025

(54) MOLECULE BEACON-BASED HYBRIDIZATION SENSOR FOR THE DETECTION OF A SINGLE NUCLEOTIDE VARIATION IN FOLDED NUCLEIC ACIDS

(71) Applicant: UNIVERSITY OF CENTRAL FLORIDA RESEARCH FOUNDATION, INC., Orlando, FL (US)

(72) Inventors: Brittany MUELLER, Orlando, FL (US); Dmitry KOLPASHCHIKOV, Orlando, FL (US); Mark LIBERMAN, Orlando, FL (US)

(21) Appl. No.: 18/756,173

(22) Filed: Jun. 27, 2024

Related U.S. Application Data
(60) Provisional application No. 63/523,420, filed on Jun. 27, 2023.

(51) Int. Cl. C12Q 1/6818; C12Q 1/6827; G01N 21/64

(52) U.S. Cl. CPC C12Q 1/6818; C12Q 1/6827; G01N 21/6428; G01N 2021/6439

(57) ABSTRACT

Hybridization probes have been used in the detection of specific nucleic acids for the last 50 years. Despite the extensive efforts and the great significance, the challenges of the commonly used probes include (1) low selectivity in detecting single nucleotide variations (SNV) at low (e.g., room or 37° C.) temperatures; (2) low affinity in binding folded nucleic acids, and (3) the cost of fluorescent probes. To address all three issues, a multicomponent hybridization probe, called OWL2 sensor, is introduced. OWL2 sensor uses two analyte binding arms to tightly bind and unwind folded nucleic acid analytes, and two sequence-specific strands that bind both the analyte and a universal molecular beacon (UMB) probe to form fluorescent ‘OWL’ structure. OWL2 sensor can differentiate single base variations in folded analytes in a temperature range of 5-38° C. The design is cost-efficient since the same optimized fluorescently labeled UMB probe can be used for the detection of any analyte sequence.

### GOVERNMENT SUPPORT CLAUSE

This invention was made with government support under cooperative agreement SHF-1907824 and CCF: SHF-2226021 awarded by National Science Foundation through the CCF: Software and Hardware Foundations. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Jan. 13, 2025, is named “10669-402US1.xml” and is 68,218 bytes in size. The sequence listing contained in this .XML file is part of the specification and is hereby incorporated by reference herein in its entirety.

### TECHNICAL FIELD

The present disclosure is related to a system for the detection of single nucleotide variations in analytes using a universal molecular beacon probe and additional oligonucleotides.

### BACKGROUND

Single nucleotide variations (SNVs) are the most common cause of genetic alterations in the human genome.¹⁻³ The identification of specific SNVs aids in the management of human genetic disorders, and early SNV detection in clinically relevant microbes is crucial in treating infections caused by drug resistant pathogens.⁴⁻⁸

Traditional methods for SNV detection include DNA sequencing, polymerase chain reaction (PCR) with melting curve analysis,⁹ and hybridization assays. DNA sequencing, such as next-generation sequencing (NGS), requires expensive instrumentation and a significant amount of time for data processing.¹⁰ PCR has an astounding range of applications from probe-based real-time PCR to post-amplification product analysis but relies on expensive instrumentation with precise temperature control for SNV differentiation.¹¹⁻¹⁴ Hybridization assays utilizing peptide nucleic acid and locked nucleic acid probes,^{15,16} cycling probe technology,¹⁷ TaqMan,¹⁸ and Molecular Beacon (MB)¹⁹ probes all suffer from the affinity/selectivity dilemma, which declares that tight binding of a probe to an analyte is associated with low selectivity.^{20,21} Recent advances in SNV detection include ratio sensing via depletion of wild-type (WT) target,²² programmable DNAzymes,²³ the use of CRISPR/Cas systems in conjunction with hybridization chain reactions,²⁴ and detection via lateral flow dipsticks after recombinase polymerase amplification with altered primers.²⁵ The best studied hybridization probes, however, all share the challenges of inefficient hybridization with RNA and DNA analytes folded in stable secondary structures, difficulty differentiating between wild-type (WT) and SNV-containing DNA at ambient temperatures, and their high synthetic cost.^{20,21,26,27}

Of the hybridization assays, the MB probe, a fluorophore- and quencher-labeled DNA harpin, has one of the most elegant designs.^{19,28} The GC rich stem enables the quencher and fluorophore to remain in proximity for more efficient quenching in the absence of the complementary analyte sequence. Upon hybridization to the complementary analyte, the MB probe opens into an elongated conformation, and fluorescence is observed.^{19,28} A typical MB probe achieves a limit of detection (LOD) of ˜1 nM,²⁸ establishing it as a significant diagnostic tool capable of detecting specific nucleic acids after amplification.²⁹⁻³¹

Although the MB probe is able to differentiate SNVs in a broader temperature range than linear (hairpin-free) probes,²⁶ they fail in differentiating SNV at ambient (0-40° C.) temperatures and, in practice, require costly instrumentation capable of measuring DNA-melting profiles.²⁹⁻³¹ Moreover, the MB probe is unable to hybridize with analytes folded in stable secondary structures because it first needs to overcome unfolding of its own stem-loop before hybridizing with another nucleic acid sequence.^{19,32}

To enable SNV differentiation at ambient temperatures, in a previous study, DNA nanotechnology was utilized, and an MB probe-based sensor was designed, which forms a four-stranded complex in the presence of an analyte, dubbed OWL sensor.³³ In the OWL structure, P and R strands hybridize to the analyte adjacent to each other and cooperatively open the MB probe hairpin, which is now called OWL1. Indeed, OWL1 sensor differentiated SNVs in the entire range of 5-32° C. with single-base mismatched analytes producing only background fluorescence.³³

Importantly, at least in part, this unprecedented SNV selectivity was attributed to the unique rigid OWL nanoscale structure: both strands P and R must fold in ‘circular’ forms with 3′- and 5′-terminal base pairs being in stacking interactions with each other, thus creating a structural lock. That is, if the probe binds a 10-nucleotide segment (which is close to the shortest possible in practice), a single base mismatch will destabilize the complex by only ˜10% on average. While increasing the length of the recognized fragment provides greater affinity, a mismatch then contributes to a proportionally lower destabilization effect, leading to even poorer SNV selectivity and differentiation. Balancing probe affinity and selectivity is a fundamental limitation of the conventional hybridization probes. That is, a rigid object fails more easily than a flexible one. Based on this idea, OWL sensor, now called OWL1, that forms a rigid and structurally imperfect nucleotide complex when it binds to a complementary SNV on the analyte was designed, OWL1 sensor. In OWL1 sensor, a single base mismatch serves the role of ‘stress’ and causes the collapse of the entire fluorescent structure, allowing the sensor to effectively differentiate between fully complementary and mismatched SNV on the analytes. This feature of the nanoscale structure makes the OWL sensor structurally constrained and less tolerant to mismatches in comparison with other hybridization probes that possess ‘unlocked’ ends (e.g. MB probe).³³

OWL1 has another advantage over previously known SNV probe. Adjusting the OWL1 sensor to each new analyte requires changing only analyte binding portion of R and P strands, while the same MB probe can be used for the analysis of any nucleic acid sequence. This allows for an opportunity to optimize only one universal MB (UMB) probe, which reduces the optimization efforts and the assay cost in comparison with the MB probe approach if multiple sequences are to be detected.

However, the OWL1 sensor structure is too ‘fragile’ to form a complex with RNA or ssDNA analytes folded in stable secondary structures.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 presents MB probe and the design of OWL1 Sensor. (FIG. 1A) MB probe (FIG. 1B) OWL1 sensor forms a 4-stranded fluorescent OWL structure only in the presence of the matched analyte. UMB (universal MB) probe is not dependent on the analyte's sequence and can be used universally.

FIG. 2 shows the design of the OWL2 sensor. OWL2 sensor consists of P strand, UMB probe, and an association of strands of T1, T2 comprising T2 arm, T3 comprising R strand, and T4 comprising T4-arm (top). The strands form a fluorescent structure, even in the presence of folded analytes (bottom).

FIG. 3 shows SNV differentiation in Tau analytes. (FIG. 3A) The secondary structures of Tau60-WT (SEQ ID NO: 24) and Tau18-WT (SEQ ID NO: 37) analytes predicted by NUPACK.³⁴ The SNV sites are circled red, and the regions of OWL2 hybridization (P/R/T2 and T4 arms) are outlined around their structure. (FIG. 3B) OWL2 sensor (UMB, 25 nM; P⁹₈, 200 nM; T1/T2/T3/T4 association 100 nM, in the hybridization buffer 1:50 mM Tris-HCl, 50 mM MgCl2, 0.1% Tween-20, pH 7.4) was incubated with 100 nM Tau60-WT (purple) or Tau18-WT (s(striped, purple) or corresponding single-base mismatched analytes (grey). The data is the average of three independent measurements. (FIG. 3C) Differentiation table for Tau60 (folded) analytes with formula for differentiation factor, Df, where ΔF represents the difference between the measured signal and the blank.

FIG. 4 shows that OWL2 equipped with P⁹₈ strand was the most selective. (FIG. 4A) OWL2 design with changes in the highlighted region depicted below the OWL2 structure. P⁹₉ contains 9 nucleotides complementary each to UMB and analyte, P⁹₈ contains 9 nucleotides complementary to UMB and 8 nucleotides complementary to analyte. C⁹₈ has the UMB- and analyte-binding arms of similar length as P⁹₈. (FIG. 4B) Fluorescence response (S/B) of OWL2 sensor containing different SNV specific stands in the presence 100 nM fully matched Tau60-WT or Tau18-WT (dark grey bars) or single base mismatched analytes (light grey bars). The data is an average of three independent measurements.

FIG. 5 shows that OWL2 sensor detects the fully matched analyte in ˜125 times excess of single-base mismatched analytes. The limit of detection of the Tau60-WT analyte in the presence of 50 nM Tau-60 0C (black line) is 0.4 nM, which is the same as the LOD in the absence of mismatch (red line) and corresponds to a detection in the presence of 125× mismatch; 100 nM OWL2 (T1/T2/T3/T4), 25 nM UMB15, 200 nM P⁹₈ in hybridization buffer 1 (50 nM and 0 nM Tau60-WT). The limit of detection of Tau60-WT analyte in the presence of 500 nM Tau60-0C (blue line) is 7.9 nM, corresponding to a detection in the presence of 60× mismatch with an increase in sensor concentrations; 600 nM OWL2 (T1/T2/T3/T4), 25 nM UMB15, 50 nM P⁹₈ strand.

FIG. 6 shows discrimination of G:T mismatches. (FIG. 6A) S/B response of the OWL2 sensor to the presence of 100 nM fully matched (M) or mismatched analytes (A:C and G:T) as indicated above the bars. The data is the average of three independent measurements. (FIG. 6B) Sequences of the P-strand (SEQ ID NOS: 17, 38 and 39, respectively) and analytes with changes in the P-strand (SEQ ID NOS: 40-43, respectively) highlighted in black and the analytes shown below, complementary to the P strand. A:C mismatches are highlighted in green and G:T mismatches are highlighted in red. (FIG. 6C) Tabulated S/B and Df values for each analyte. Tau60-WT is denoted “WT” in the table, but it is only fully complementary to the normal (unsubstituted) P⁹₈.

FIG. 7 shows that OWL2 sensor differentiates SNVs in Covid-19-related sequences. (FIG. 7A) OWL2 sensor adapted for detection of Covid-19 analyte; T1 and UMB remain unchanged. (FIG. 7B) S/B for OWL2 sensor with 200 nM CP⁹₈ and CP⁹₉ in the presence of 100 nM analyte. (FIG. 7C) (SEQ ID NO: 34) Secondary structure of Covid19-WT analyte used in this study. T>G and T>C mutations are indicated by red circles. (D) Values for S/B and Differentiation Factor (D_{f}) for each analyte. The data is average three independent measurements.

FIG. 8. Optimization of P-strand concentration for the OWL2 sensor equipped with P9 8. FIG. 8A) Response of the sensor containing different concentrations of P 9 8 and 100 nM other sensor's components. FIG. 8B) Table depicting the exact values for S/B from the graph, as well as differentiation factor calculated as Df=1−ΔFₘₘ/ΔFₘ, where ΔF represents the difference between the signal and the blank for the mismatched (mm) and matched (m) analyte, respectively.

FIG. 9. OWL1 and OWL2 sensors in complex with Tau analyte and LOD for the OWL1 sensor. FIG. 9A) (SEQ ID NOS: 1 (gold), 44 (brown), 17 (orange) and 31 (pink)) OWL1 design consists of R- and P-strand along with UMB-15 to form a complex with a short Tau18-WT analyte. FIG. 9B) Limit of detection for the OWL1 sensor using Tau18-WT. FIG. 9C) (SEQ ID NOS: 3 (navy), 5 (brown), 9 (green), 2 (teal), 1 (gold), 17 (orange), 24 (pink)) OWL2 design has additional T2- and T4-unwinding arms which allow for the opening of a longer Tau60-WT analyte folded into a stable secondary structure.

FIG. 10. Melting curve for the OWL2 sensor shows differentiation between the fully matched and SNV containing analytes over a temperature range of 5-38° C. FIG. 10A. The melting curve normalized using ROX as an internal reporter shows a higher signal triggered by Tau60-WT than by unstructured Tau18-WT, which correlates to the data in FIG. 3B. FIG. 10B. The ratio of the OWL2 sensor's signal triggered by the fully matched Tau60-WT analyte to the signal in the presence of the indicated mismatched Tau60 analytes. The dashed 1.5 line is the threshold at which we determine that the wild-type analyte has been differentiated from the mutant.

FIG. 11. Flexible linkers between the R stand and scaffold enable higher analyte-triggered signal. FIG. 11A (SEQ ID NOS: 3 (navy), 5 (brown), 7 (green), 2 (teal), 1 (gold), 17 (orange), 24 (pink)) OWL2 design with P⁹₉ with the highlighted region representing the linker between T1-hybridizing portion of T3 and the UMB- and analyte-hybridizing portion of T3 FIG. 11B Fluorescence measured on PerkinElmer Fluorimeter showing an increase in fluorescence for all analytes when flexible linkers were introduced in the T3 strand. FIG. 11C (SEQ ID NOS: 45-47, 53) Table containing the sequence of T3 and the types of linkers tested.

FIG. 12 shows that introduction of a gap between P⁹₈ and T4 arm does not significantly destabilize the OWL structure. For T4, two strands were used; one in which there is no gap between the T4 arm and P⁹₈ (denoted with an asterisk) and one in which there is an introduction of a gap (denoted as T4-1) between P-strand and T4. FIG. 12A (SEQ ID NOS: 3 (navy), 5 (brown), 9 (green), 2 (teal), 1 (gold), 17 (orange), 24 (pink)) Design of OWL2 in complex with Tau60 WT, P⁹₈, and UMB. The linker variation is the highlighted region between T3 and R. The highlighted nucleotide in T4 arm represents the nucleotide that is removed to introduce a gap between P-strand and T4 arm (T4-1). FIG. 12B The fluorescent readout from PerkinElmeer LS55 Fluorimeter showing that a more flexible linker leads to a higher signal with insignificant compromise to differentiation. FIG. 12C The limit of detection for the OWL2 sensor with ttt and iSp18 linker and T4 arm is 0.35 nM, which is comparable to the 0.26 nM LOD for the ttt linker. FIG. 12D Signal to background and Differentiation factor for the OWL2 variations shown in FIG. 12B. FIG. 12E (SEQ ID NOS: 4-6) T3 sequences corresponding to the different linkers.

FIG. 13 shows that the constrained structure of P strand contributes to high selectivity of OWL2 sensor. FIG. 13A OWL2 design with changes in the highlighted region depicted below the OWL2 structure. R¹⁰₁₀ with an internal iSp18 linker was used in conjunction with P⁹₈, which contains 9 nucleotides complementary to UMB and 8 nucleotides complementary to analyte. P⁹₉ contains 9 nucleotides complementary each to both UMB and analyte. C⁹₈ has similar binding to P⁹₈, but nucleotides complementary to UMB are consecutive starting at the 5′-end and nucleotides complementary to analyte are consecutive ending at the 3′-end. FIG. 13B Fluorescence for P⁹₉ is higher for Tau60 WT than for P⁹₈ but has diminished differentiation of mutations. Fluorescence for C⁹₈ is comparable to that of P⁹₉ but has poorer differentiation due to the flexibility of the C-strand. See table FIG. 6(C).

FIG. 14 shows that removal of a nucleotide on T4 to introduce a gap between P and T4 has no effect on fluorescence or limit of detection. FIG. 14A (SEQ ID NOS: 3 (navy), 5 (brown), 9 (green), 2 (teal), 1 (gold), 17 (orange), 24 (pink)) OWL2 design in complex with UMB, P⁹₈, and analyte. Removed nucleotide on T4 is FIG. 14B Fluorescence measured on PerkinElmer LS55 Fluorimeter with no appreciable difference between the two designs. FIG. 14C Limit of Detection created using Fluorescence read on a Perkin-Elmer Fluorimeter with a LOD of 0.22 nM for T4-1 (removed nucleotide) design and a LOD of 0.32 nM for the original OWL2 design.

FIG. 15 presents the introduction of a single gap between the T4 arm and the P strand. FIG. 15A (SEQ ID NO: 1) The introduction of a gap between P-strand (SEQ ID NO: 17) and T4 arm (SEQ ID NO: 48) via removal of a nucleotide on the T4 arm slightly destabilizes the OWL2 Structure. FIG. 15B. The S/B for fully matched analyte (SEQ ID NO: 24) decreases upon the addition of a gap, except in the case of P¹⁰₈. It is thought that the increase in signal upon introduction of a gap is due to the sequence of the P-strand; when there is a gap, the first nucleotide intended to hybridize with the UMB, underlined in FIG. 15C (SEQ ID NOS: 15-20), may instead hybridize with the analyte since they share complementarity.

FIG. 16 (SEQ ID NOS: 24, 25, 26 and 27) presents secondary structures and free energies of Tau analytes. FIG. 16A Tau WT with T2-binding arm outlined in blue, T3-R-binding in brown, P-binding in orange, and T4-binding arm in green. SNP-containing mutants of Tau with mutations 0C FIG. 16B, 1A (FIG. 16C, and 2G FIG. 16D are indicated with a red arrow.

FIG. 17 (SEQ ID NOS: 34, 36 and 35) presents secondary structures of covid and mutant covid. FIG. 17A Covid WT with T2-binding arm outlined in blue, T3-R-binding in brown, P-binding in orange, T4-binding arm in green, and SNP-locations highlighted in yellow. FIG. 17B and FIG. 17C SNP-containing mutants of covid with mutations are indicated with a red arrow.

FIG. 18 presents fluorescent response of OWL2 sensors over time for Tau analytes. Fluorescence was read over 45 min for each mismatch. A four-fold increase in fluorescence can be seen over the first ten minutes with discrimination against mutants 0C and 1A. The 2G mismatch shows a slower increase when compared to the WT.

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### Definitions

In describing and claiming the disclosed subject matter, the following terminology will be used in accordance with the definitions set forth below.

The terms “oligonucleotide” and “polynucleotide” as used herein refer to any polyribonucleotide or polydeoxyribonucleotide that may be unmodified RNA or DNA or modified RNA or DNA. Thus, for instance, oligonucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid,” “nucleic acid sequence,” or “oligonucleotide” also encompass a polynucleotide as defined above.

The term “nucleotide” as used herein refers to a sub-unit of a nucleic acid (whether DNA or RNA or an analogue thereof) which may include, but is not limited to, a phosphate group, a sugar group and a nitrogen containing base, as well as analogs of such sub-units. Other groups (e.g., protecting groups) can be attached to the sugar group and nitrogen containing base group. It will be appreciated that, as used herein, the terms “nucleotide” and “nucleoside” will include those moieties which contain not only the naturally occurring purine and pyrimidine bases, e.g., adenine (A), thymine (T), cytosine (C), guanine (G), or uracil (U), but also modified purine and pyrimidine bases and other heterocyclic bases which have been modified (these moieties are sometimes referred to herein, collectively, as “purine and pyrimidine bases and analogs thereof”). Such modifications include, e.g., diaminopurine and its derivatives, inosine and its derivatives, alkylated purines or pyrimidines, acylated purines or pyrimidines thiolated purines or pyrimidines, and the like, or the addition of a protecting group such as acetyl, difluoroacetyl, trifluoroacetyl, isobutyryl, benzoyl, 9-fluorenylmethoxycarbonyl, phenoxyacetyl, dimethylformamidine, N,N-diphenyl carbamate, or the like. The purine or pyrimidine base may also be an analog of the foregoing; suitable analogs will be known to those skilled in the art and are described in pertinent texts and literature. Common analogs include, but are not limited to, 1-methyladenine, 2-methyladenine, N6-methyladenine, N6-isopentyladenine, 2-methylthio-N6-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl) uracil, 5-(methylaminomethyl) uracil, 5-(carboxymethylaminomethyl)-uracil, 2-thiouracil, 5-methyl-2-thiouracil, 5-(2-bromovinyl) uracil, uracil-5-oxyacetic acid, uracil-5-oxyacetic acid methyl ester, pseudouracil, 1-methylpseudouracil, queuosine, inosine, 1-methylinosine, hypoxanthine, xanthine, 2-aminopurine, 6-hydroxyaminopurine, 6-thiopurine, and 2,6-diaminopurine.

The term “hybridization” as used herein refers to the process of association of two nucleic acid strands to form an anti-parallel duplex stabilized by means of hydrogen bonding between residues of the opposite nucleic acid strands. The terms “hybridizing” and “binding”, with respect to polynucleotides, are used interchangeably and is meant the formation of A-T and C-G base pairs between the nucleotide sequence of a fragment of a segment of a polynucleotide and a complementary nucleotide sequence of an oligonucleotide. By complementary is meant that at the locus of each A, C, G or T (or U in a ribonucleotide) in the fragment sequence, the oligonucleotide sequence has a T, G, C or A, respectively. The hybridized fragment/oligonucleotide is called a “duplex.”

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “complementary” as used herein refers to a sufficient number in the oligonucleotide of complementary base pairs in its sequence to interact specifically (hybridize) with the target nucleic acid sequence to be amplified or detected. As known to those skilled in the art, a very high degree of complementarity is needed for specificity and sensitivity involving hybridization, although it need not be 100%. Thus, for example, an oligonucleotide that is identical in nucleotide sequence to an oligonucleotide disclosed herein, except for one base change or substitution, may function equivalently to the disclosed oligonucleotides. A “complementary DNA” or “cDNA” gene includes recombinant genes synthesized by reverse transcription of messenger RNA (“mRNA”).

The terms “analyte” and “target analyte” as used herein refer to an oligonucleotide or polynucleotide for which it is desired to detect. The analyte for use in the methods herein disclosed may be an oligonucleotide or a polynucleotide, immobilized on a solid support or in free solution, which is isolated from a plant or animal host, a cultured cell or a cell or population of cells in a tissue of a plant or animal.

The terms “probe” as used herein refers to an oligonucleotide of nucleic acid sequences of variable length for the detection of identical, similar, or complementary nucleic acid sequences by hybridization.

The term “molecular beacon” and “oligonucleotide molecular beacon” as used herein refer to a form of an oligonucleotide having a fluorophore- and quencher-moiety attached stem-loop structure of oligo-deoxyribonucleotide that is widely used for real-time detection of specific RNA/DNA sequences. In the stem-loop conformation, a fluorophore is brought close to the quencher enabling efficient fluorescent quenching. Hybridization to a complementary DNA/RNA target analyte switches the molecular beacon conformation to an elongated form, which is accompanied by the fluorescence increase.

The term “fluorophore” as used herein refers to any fluorescent dye group whose presence can be detected by its light absorbing or light emitting properties. For example, Cy5 is a reactive water-soluble fluorescent dye of the cyanine dye family. Cy5 is fluorescent in the red region (about 650 to about 670 nm). It may be synthesized with reactive groups on either one or both of the nitrogen side chains so that they can be chemically linked to either nucleic acids or protein molecules. Labeling is done for visualization and quantification purposes. Cy5 is excited maximally at about 649 nm and emits maximally at about 670 nm, in the far-red part of the spectrum; quantum yield is 0.28. FW=792. Suitable fluorophores (chromes) for the probes of the disclosure may be selected from, but not intended to be limited to fluorescein amidite, fluorescein isothiocyanate (FITC, green), 4,5,6,7-tetrachlorofluorescein, 6-carboxy-2′,4,4′,5′,7,7-hexachlorofluorescein, cyanine dyes Cy2, Cy3, Cy3.5, Cy5, Cy5.5 Cy7, Cy7.5 (ranging from green to near-infrared), Texas Red, rhodamine 123 (hydrochloride), sulforhodamine 101 acid chloride succinimidyl ester, 2-3-(dimethylamino)-6-dimethyliminio-xanthen-9-ylbenzoate, (2E)-2-(2E,4E)-5-(2-tert-butyl-9-ethyl-6,8,8-trimethyl-pyrano 3,2-gquinolin-1-ium-4-yl) penta-2,4-dienylidene-1-(6-hydroxy-6-oxo-hexyl)-3,3-dimethylindoline-5-sulfonate, and the like. Derivatives of these dyes for use in the embodiments of the disclosure may be, but are not limited to, Cy dyes (Amersham Bioscience), Alexa Fluors (Molecular Probes Inc.), HILYTE® Fluors (AnaSpec), and DYLITE® Fluors (Pierce, Inc).

The terms “quench” or “quenches” or “quenching” or “quenched” as used herein refer to reducing the fluorescence signal produced by a molecule. It includes, but is not limited to, reducing the signal produced to zero or to below a detectable limit. Hence, a given molecule can be “quenched” by, for example, another molecule and still produce a detectable signal albeit the intensity of the signal produced by the quenched molecule is smaller when the molecule is quenched than when the molecule is not quenched.

The terms “fluorescence quencher” or “quencher” as used herein refer to a molecule that interferes with the fluorescence emitted by a fluorophore. Quenchers prevent the emission of fluorescence when they are physically near a fluorophore. This quencher can be selected from non-fluorescent dark quenchers, which are substances that absorb excitation energy from fluorophores and dissipate the energy as heat. Exemplary dark quenchers include, but are not limited to, such as Dabsyl (4-((4′-dimethylamino)phenylazo)benzoic acid), BLACK HOLE QUENCHER®, IOWA BLACK®. The quencher can also be, but is not limited to, a fluorescent molecule, for example TAMRA (carboxytetramethylrhodamine). When the quencher is a fluorescent dye, its fluorescence wavelength is typically substantially different from that of the reporter dye and the quencher fluorescence is usually not monitored during an assay.

The term “solid support” is used herein refers to any insoluble and inert inorganic or organic material, preferably having a large surface area to which surface organic molecules can be attached through bond formation or absorbed through electronic or static interactions. Representative examples of a “solid support” in context with the present invention are silicates, such as SiO2 resin, including, but not limited to, ion-exchange resins, glass, dextranes, celluloses or hydrophilic or hydrophobic polymers.

By “immobilized on a solid support” is meant that an oligonucleotide is attached to a substance at a particular location in such a manner that the system containing the immobilized fragment, primer or oligonucleotide may be subjected to washing or other physical or chemical manipulation without being dislodged from that location. A number of solid supports and means of immobilizing nucleotide-containing molecules to them are known in the art; any of these supports and means may be used in the methods of this disclosure.

Further definitions are provided in context below. Unless otherwise defined, all technical and scientific terms used herein have the same meaning as commonly understood by one of ordinary skill in the art of molecular biology. Although methods and materials similar or equivalent to those described herein can be used in the practice or testing of the present disclosure, suitable methods and materials are described herein.

### Introduction

The developments of DNA nanotechnology have been applied to designing smart hybridization sensors that can simplify sequence specific analysis of nucleic acids. An ideal hybridization sensor should be able to detect a targeted sequence within the context of folded RNA or dsDNA with selectivity close to absolute (measured as Df near 1 in this study) and under broad experimental conditions including all practically significant temperatures. The probe should be affordable and easily adjustable to each new analyte sequence. Toward satisfying these requirements, OWL2 sensor was developed. The OWL2 sensor shines where most hybridization probes fall short. The remarkable characteristics of the sensor include a S/B of 18 and LOD in sub-nanomolar range both for DNA and RNA analytes. It has an extraordinary ability to differentiate mismatched analytes from the fully matched ones including the most challenging G-T mismatches in the temperature range of 5-38° C. Despite looking complex, the sensor is cost efficient when applied for new analytes. The UMB reporter, the most expensive and hard to design component, is analyte independent, so that can be optimized once and then used for the analysis of any DNA or RNA sequences. These features make the OWL2 sensor a highly specific, selective, and versatile tool, which seeks to improve the field of hybridization assays.

### Overview

The OWL2 sensor was able to tightly bind analytes folded in secondary structure as stable as −8.76 kcal/mol. The achieved signal turn-on ratio of ˜18 were comparable with that of a typical MB probe detecting a linear analytes and exceeded that of the earlier reported OWL1 sensor by ˜6 times. Measured LOD of 0.25 nM exceeded that of MB probe (˜1 nM) and OWL 1 sensor (˜6 nM).³³ The improvements in S/B and LOD are the consequences of the tight analyte binding by T2 and T4 arms, which increased the local concentration of the analyte near R strand, which also contributes to stabilization of the entire OWL structure.

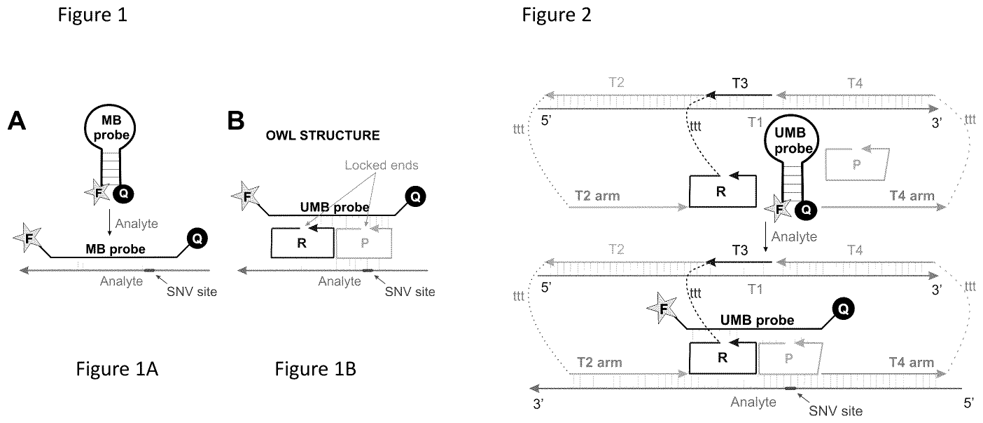

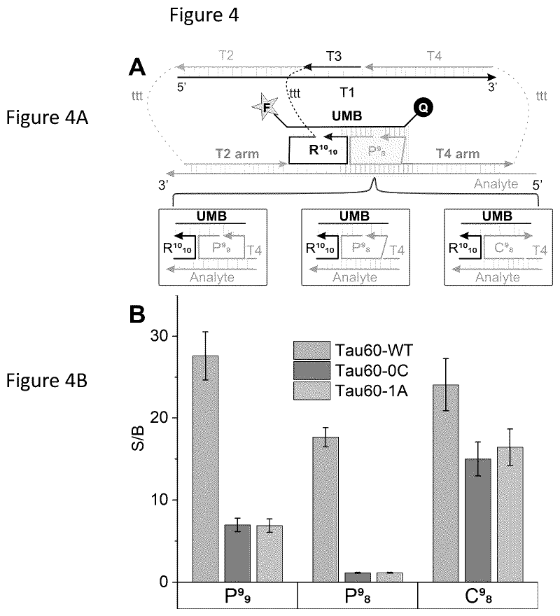

The outstanding quality of OWL2 sensor is its selectivity to SNV in the broad temperature range of 5-38° C. Such a broad range cannot be explained neither by the thermodynamic considerations or by the kinetic inversion phenomenon.³³ It has been hypothesized that such performance is the consequence of the rigid structure of the OWL complex (FIG. 1B). In OWL structure, 3′ and 5′ terminal base pairs of strand P are in stacking interactions, which restricts their movement thus introducing structural constraint. To verify this hypothesis, stand P was replaced with strand C having the same analyte binding arm, but unstacked 5′ and 3′ terminal vase pairs. Such open sensor produced higher S/B but demonstrated significant reduction in ability to differentiate SNV (FIG. 4). This data correlate with our earlier observations that flexibility introduced in P strand in the form of ethylene glycol linkers reduced SNV differentiation of OWL1 sensor.³³

The OWL2 design seems more complex than ‘straightforward’ design of MB probe. In practice, the design of MB probe is associated with loop and stem interferences problems to the extent that it is impossible to design classical MB probes for a broad range of unfolded sequences let alone for folded nucleic acids. OWL2 design can accommodate any MB sequence, which opens a possibility to optimize one universal MB probe and use it for all analytes. This makes OWL2 technology more practical than MB probe technology.

In OWL2 technology, to adjust OWL sensor to each new analyte, only changing the sequences of 4 portions (T2-arm of T2 strand T4-arm of T4 strand, R strand of T3 strand), and P strand to each new analyte is required. In addition, according to Integrated DNA technologies Inc. prices, the total cost of the four DNA strands is $34.06 (minimal synthetic scale). At the same time, the cost of a new MB probe at the minimal synthetic scale is $250.00, or 7.33 times more. Overall, this makes OWL2 more affordable for research and teaching communities tank MB probe, which can save resources on MB optimization stage and if multiple analytes are to be detected.

### Exemplary Embodiments

### (1) OWL Sensor System

To enable SNV differentiation at ambient temperatures, DNA nanotechnology was used to design a molecular beacon (MB) probe-based sensor, which forms a four-stranded complex in the presence of an analyte, dubbed OWL sensor³³ (OWL1 sensor in FIG. 1B).

As shown in FIG. 1A, an MB probe has a hairpin (or stem-loop) structure having a fluorophore at one end and a quencher at the other end. In the absence of an analyte, the MB is of a closed form, in which fluorescence emitted by the fluorophore is decreased by the quencher in proximity; however, in the presence of an analyte hybridizing to the oligonucleotide of the MB probe, the hairpin structure changes to an elongated form, the fluorophore and the quencher become distant, and fluorescence can be detected free from the effect of the quencher.

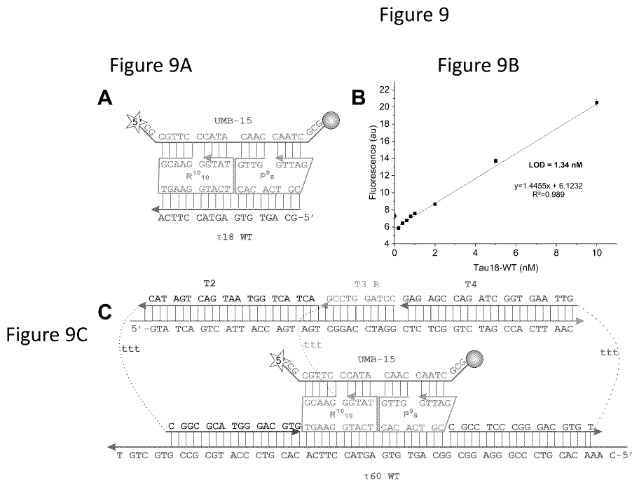

FIG. 1B and FIG. 9A illustrate the basic design of OWL1 sensor. OWL1 sensor comprises two DNA adaptor strands R and P, and a universal molecular beacon (UMB) probe. The UMB probe does not directly bind to the analyte and therefore can be used for analysis of any sequences given that the adaptor strands are adjusted accordingly. The adaptor strands are named R^{x}_{y} and P^{x}_{y}, where x indicates the number of nucleotides in the UMB probe binding domain of the R or P strand, and y indicates the number of nucleotides in the analyte-binding domains. The central portions of the adaptor strands are complementary to the analyte and are thus called analyte-binding domains, while the 4- to 5-nt long 3′ and 5′ terminal sequences are complementary to the UMB. In the presence of a specific analyte, R and P strands bind to both UMB and the analyte, thus forming a 4-stranded fluorescent complex, which, when drawn, resembles owl eyes, suggesting the name of the structure and the sensor.

R and P strands hybridize to two regions close or adjacent to each other on the analyte, respectively, and on the oligonucleotide of MB probe, respectively, at the same time to cooperatively open the MB probe hairpin structure. P strand has only 9-10 nucleotides complementary to the analyte. Therefore, a single base mismatch would destabilize the hybridization between the P strand and the analyte. That is, without P strand, which has a complementary nucleotide to the SNV of the analyte, the fluorophore and quencher are still in proximity even in the presence of R strand and the analyte, and as an MB probe is folded, only basal level of fluorescence is generated. However, when P strand fully binds to the SNV of the analyte, the MB probe is unfolded and gives enough distance between the fluorophore and the quencher to allow the fluorescence to be generated, which allows for the detection of the desired sequence. Indeed, OWL1 sensor differentiated SNVs in analytes in the entire range of 5-32° C., producing only the background florescence.

Importantly, at least in part, this unprecedented SNV selectivity was attributed to the unique rigid structure of the OWL1 complex: for the generation of fluorescence signals, both P and R strands must fold in circular forms with 3′ and 5′ terminal base pairs being in stacking interactions with each other, i.e., ‘locked’ ends in which the 5′-terminus and 3′ terminus of a strand are parallelly forming base pairs with opposing nucleotides on the oligonucleotide of an MB probe. This feature makes OWL1 sensor structurally constrained and less tolerant to mismatches in comparison with other hybridization probes that possess ‘unlocked’ ends (e.g., MB probe in FIG. 1A).

In addition, OWL1 sensor has another advantage that for the adjustment of OWL1 sensor to a new analyte, only analyte binding regions of P and R strands are to be changed, while the same MB probe can be used for analysis of any nucleotide sequences. This opens an opportunity to optimize only one universal MB (UMB) probe for use in the analysis of SNVs in multiple analytes. This reduces the optimization efforts and the assay cost in comparison with MB probe approach if multiple analytes are to be detected.

However, OWL1 sensor structure is too ‘fragile’ to form a complex with RNA or ssDNA analytes which are folded in stable secondary structures. Therefore, a new application approach of OWL sensor system is needed, especially for folded nucleic acids.

### (2) OWL2 Sensor System

To this end, a new OWL sensor system, OWL2 sensor (FIG. 2 and FIG. 9C) is provided here. Like OWL1 sensor, OWL2 sensor uses a pair of R and P strands for binding to the oligonucleotide of a UMB probe and an analyte. However, it has additional T1, T2, T3, and T4 oligonucleotide strands as well as T2- and T4 oligonucleotide arms tethered to the T2 and T4 strands, respectively, which are for additional binding to the analyte around the R and P binding regions in order to unwind the secondary structures of the analyte.

The oligonucleotide R strand comprises: (i) at each of a 5′-terminus and 3′-terminus thereof, a region having a nucleotide sequence of 4-6 nucleotides, respectively, preferably a total of 10 nucleotides, which is complementary to a nucleotide sequence of a first region of an oligonucleotide of a UMB probe; and (ii) between the 5′-terminus and 3′-terminus thereof, a region having a nucleotide sequence of 8-12 nucleotides, preferably 10 nucleotides, which is complementary to a nucleotide sequence of a first region of an analyte. However, in OWL2 sensor, the R strand is covalently linked to the T3 strand via at least one linker or is extended from the T3 strand without any linker, i.e., the T3 strand can be connected at its 3′-terminus to the 5′ terminus of the R strand, or at its 5′-terminus to the 3′ terminus of the R strand, which is described below.

The oligonucleotide P strand comprises: (i) at each of 5′-terminus and 3′-terminus thereof, a region having a nucleotide sequence of 4-6 nucleotides, respectively, preferably a total of 9 nucleotides, which is complementary to a nucleotide sequence of a second region of the oligonucleotide of the UMB probe; and (ii) between the 5′-terminus and 3′-terminus thereof, a region having a nucleotide sequence of 8-12 nucleotides, preferably 8-9 nucleotides, which is complementary to the nucleotide sequence of a second region of an analyte, which has at least one SNV.

The UMB probe comprises (i) an oligonucleotide comprising: a first region having a nucleotide sequence complementary to the 5′-terminus and 3′-terminus of the R strand; and a second region having a nucleotide sequence complementary to the 5′-terminus and 3′-terminus of the P strand; and comprises (ii) a fluorophore at one end of the oligonucleotide and a quencher at the other end. In this disclosure, an oligonucleotide of SEQ ID NO:1 is presented as an example of an oligonucleotide of UMB probe for OWL2 sensor, however any proper length of oligonucleotides can be used for the same purpose.

The fluorophore can be a single fluorophore or combination of at least 2 fluorophores to make individual tags for different kinds of SNVs, and the quencher is selected according to the selected fluorophore.

As shown in FIG. 2 and FIG. 9C, the 4-stranded complex similar to that of OWL1 sensor composed of R and P strands, UMB probe, and the analyte is associated with a DNA scaffold platform of T1 hybridized to T2, T3, T4 strands via T2- and T4 arms and R strand extended from the T3 strand without any linker or linked to the T3 strand with at least one linker.

T1 strand is an oligonucleotide having about 50-60 nucleotides, preferably 52 nucleotides, for example, an oligonucleotide of SEQ ID NO:2 (5′-GTA TCA GTC ATT ACC AGT AGT (21 nts)-CGGAC CTAGG (10 nts) CTCT CGGT CTAG CCAC TT AAC-3′ (21 nts)), which is a nucleotide sequence complementary to T2, T3 and T4 strands. T2, T3, and T4 strands hybridize to T1 strand; for example, T2 strand hybridizes to the 21 nucleotides of the 5′-terminus of the T1 strand, T3 strand to the 10 nucleotides in the middle, and T4 strand to the 21 nucleotides of the 3′-terminus. This 5′-T1-3′/(3′-T2. T3, T4-5′) hybridization DNA fragment functions as a DNA scaffold platform in OWL2 sensor. Although in the embodiments of the disclosure, T1 strand of SEQ ID NO:2, T2 strand of 5′-ACT ACT GGT AAT GAC TGA TAC-3′ (SEQ ID NO: 49), T3 strand of 5′-CCTAG GTCCG-3′ (SEQ ID NO: 50), and T4 strand of 5′-GTT AA GTGG CTAG ACCG AGAG-3′ (SEQ ID NO: 51) are provided in Table 2 as examples, these are just exemplary nucleotide sequences, and any other nucleotide sequences with proper nucleotide length may be used for the same purpose.

In addition, any of T1, T2, T3 or T4 strands, in particular T1 strand may be biotinylated and used as an anchor to attach the T1/(T2, T3, T4) hybridization fragment and OWL2 sensor to a streptavidin-coated substrate or beads for microarray analysis or individual SNV analysis.

### (3) T2 and T4 Arms

As illustrated in FIG. 2 and FIG. 9C, the T1/(T2, T3, T4) hybridization fragment is associated with the 4-stranded complex through T2 and T4 arms having each 10-30 nucleotides, whose function is to bind regions outside the R and P binding regions on the analyte and open a secondary structure of single stranded DNA or RNA.

T2 strand comprises at its 3′-end an oligonucleotide arm (T2 arm), wherein the T2 arm is optionally connected via a linker such as at least one tri-thymidine (ttt) or hexaethylene glycol linker, and the T2 arm hybridizes to a region on the analyte, which locates 3′ direction out of the R and P strand binding regions on the analyte.

T4 strand comprises at its 5′-terminus an oligonucleotide arm (T4 arm), wherein the T4 arm is optionally connected via a linker such as at least one tri-thymidine (ttt) or hexaethylene glycol linker, and the T4 arm hybridizes to another region on the analyte, which locates 5′ direction out of the R and P strand binding regions on the analyte.

In this structure, the R strand binding region on the analyte can be either 3′ location or 5′ location relative to the P strand binding region on the analyte.

The nucleotide sequences of T2 and T4 arms, which are to be determined according to the nucleotide sequences on the analyte, are chosen to have melting temperatures above the assay/room temperature (24° C.) and to have little or no secondary structures to ensure tight analyte association with the tile.

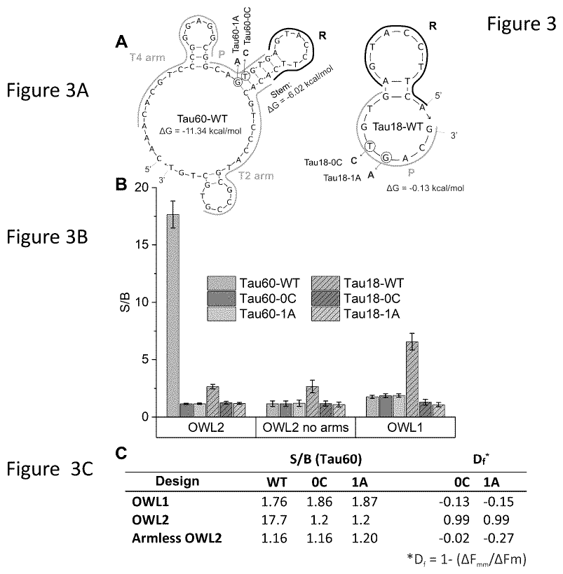

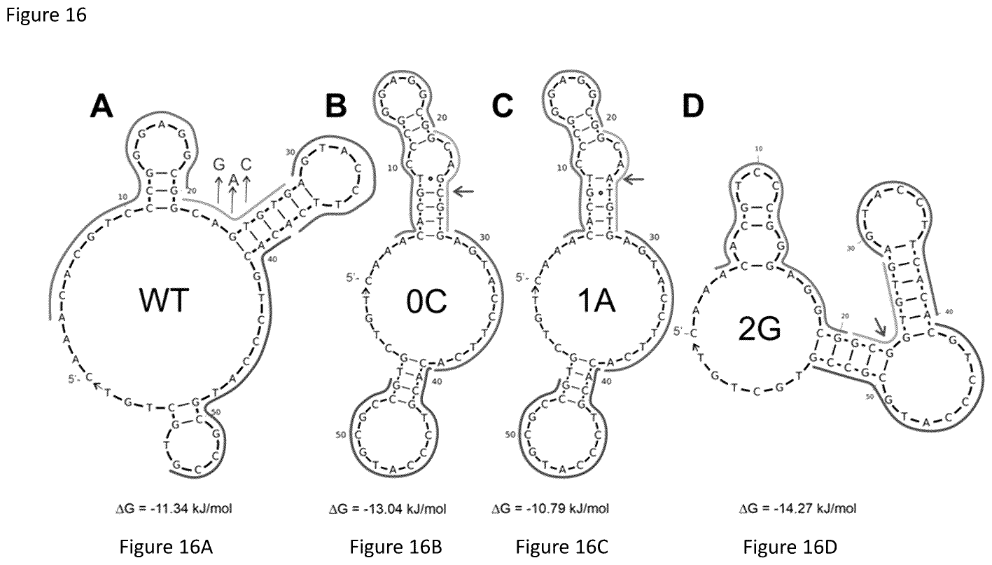

As a model analyte to demonstrate the function of T2 and T4 arms, synthetic analytes of tau gene, Tau60-WT and its SNVs named ‘0C’ and ‘1A’ were used. FIG. 3A and FIG. 16 show the secondary structure of the synthetic analytes used here. Tau60-WT has folded structures in T2- and T4 arm binding regions on either side of the P and R strand binding regions. Tau18-WT is another synthetic analyte of short oligonucleotide, having all the nucleotides complementary to P and R strands, but without T2 and T4 arm binding regions, and has weak stem-loop structure in R and P strand binding regions, thus resembling a linear analyte (FIG. 3A) under the experimental conditions.

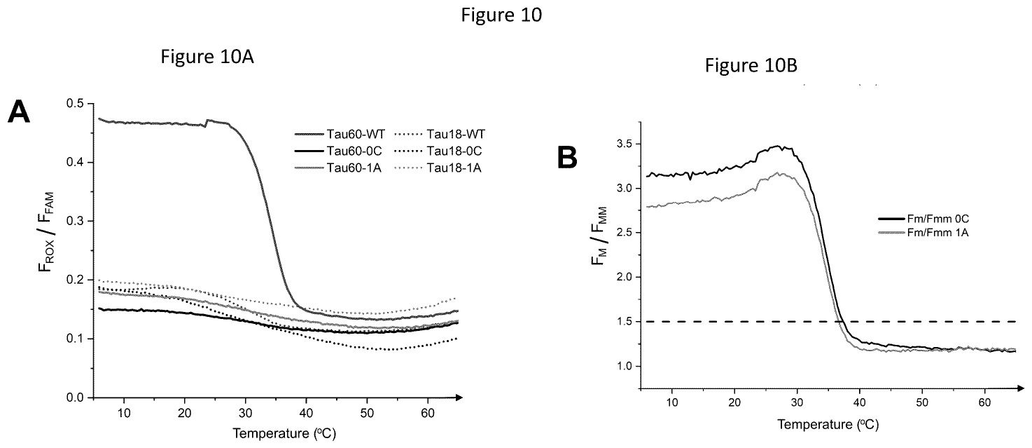

The OWL2 sensor produced fluorescence (signal to background (S/B)) of ˜18 with Tau60-WT, but fluorescence (S/B) of basal level with Tau60 SNVs (FIG. 3B); the differentiation factors (D_{f}) were 1.0 and 0.99 for Tau60-0C and 1A, respectively, where D_{f}=1.0 reflects the greatest possible selectivity. FIG. 10 presents melting curves of Tau60-WT and its SNVs probed with OWL2 sensor, showing that the OWL2 sensor can differentiate Tau60-WT from single base mismatched Tau60-1A and -0C in the temperature range of 5-38° C., which is a range ˜6° C. broader than that reported earlier for OWL1 sensor. In addition, the concentration of the limit of detection (LOD) of OWL2 sensor was ˜0.25 nM, which was one order of magnitude lower than that of OW1 sensor (˜5 nM).³³

OWL2 sensor without T2 and T4 arms as well as OWL1 failed to detect Tau60-WT (FIG. 3B), suggesting that the binding of the T2 and T4 arms to Tau60-WT decreased the energy barrier for hybridization between the R and P strands of OWL2 sensor and the analyte, and opened the secondary structure in the R and P binding regions of the analyte, which was not detected by OWL1 or OWL2 without T2 and T4 arms. Therefore, it can be concluded that the T2 and T4 arms are necessary for the detection of SNVs in a stably folded secondary structure.

Interestingly, OWL2 sensor and OWL2 sensor without T2 and T4 arms produced ˜8 times lower fluorescence (S/B) with Tau18-WT that has no T2 and T4 arm binding region than with Tau60-WT, and this indicates low level of partial unfolding of UMB and incomplete binding of P and R strands to the analyte, supporting the hypothesis that the binding of T2 and T4 arms to the analyte is important in removing the structural constrain. In addition, it may be explained that T2 and T4 arms may help localizing the analyte at the right position, which overall stabilizes the bulky OWL structure.

On the other hand, OWL1 sensor produced greater fluorescence (S/B) than OWL2 sensor with Tau18-WT (FIG. 3B). This can be explained by the reduced attraction of Tau18-WT to the bulky OWL2 sensor complex due to the electrostatic repulsion.

### (4) Flexible Linker Between R Stand and T3 Strand

As mentioned above, T3 strand is connected to R strand, and there can be optionally a linker such as tri-thymidine or hexaethylene glycol at the connection point so as to give flexibility between those two strands since the accommodation of the fragile OWL structure of bulky DNA scaffold platform in OWL2 sensor system might be challenging due to the steric hindrance.

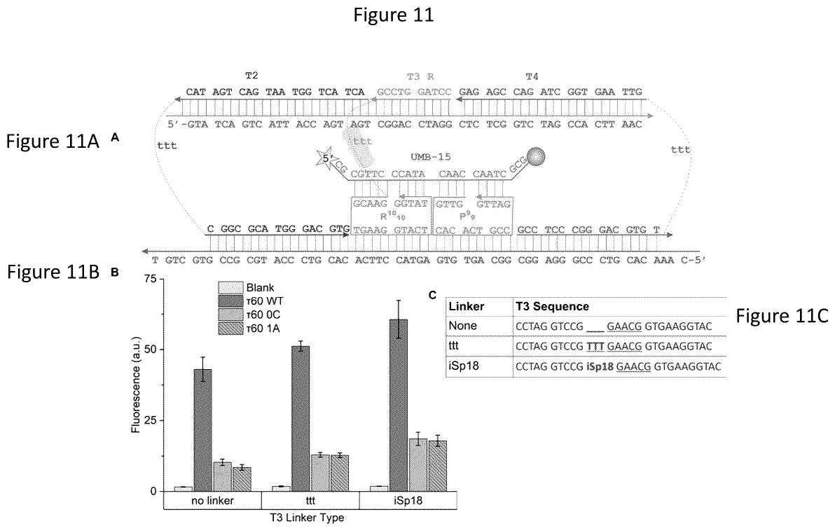

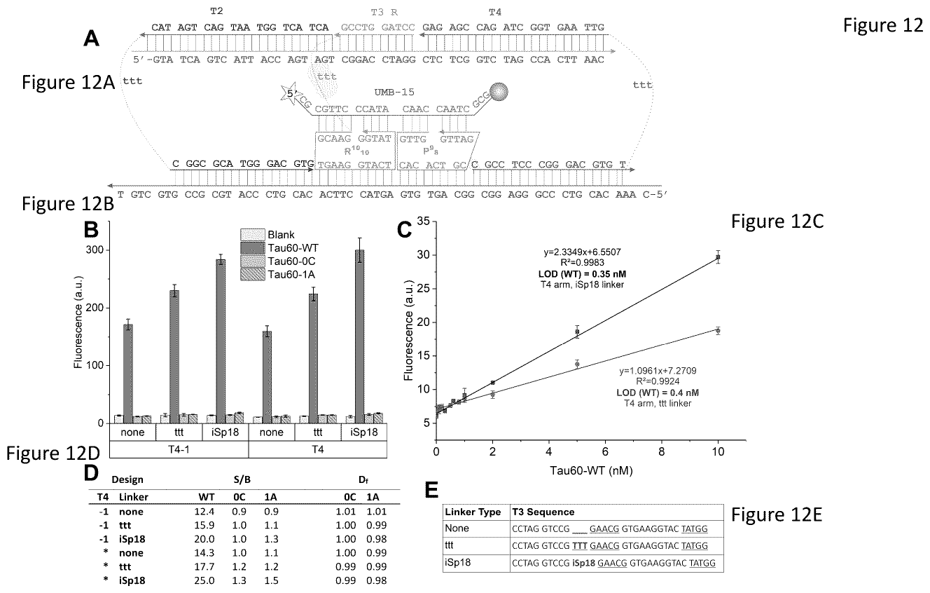

FIG. 11 shows the results of the tested linkers between R strand and the T3 strand e.g., the least flexible regular phosphodiester (PDE (none)), more flexible tri-thymidine (ttt), and the most flexible hexaethylene glycol (iSp18) linker (FIGS. 11 and 12). With P strand P⁹₉ (9 base pairs with MB probe and 9 base pairs with the analyte), the inclusion of a flexible linker increased fluorescence in Tau60-WT and more in SNVs-containing analytes of Tau60, reducing SNV differentiation (FIG. 11B). With P⁹₈ (9 base pairs with the UMB probe and 8 base pairs with the analyte), the inclusion of a flexible linker increased fluorescence in Tau60-WT with no change in SNV differentiation (FIG. 12B). However, although iSp18 increased fluorescence (S/B) more than ttt (˜25-fold vs. ˜18-fold; FIG. 12B), considering that 25-fold increase by iSp18 linker is a minor advantage over 18-fold increase by ttt linker in comparison with the lower synthetic cost of the ttt linker, majority of the experiments in this disclosure were conducted using ttt linkers between R strand and the tile-forming fragment of T3 strand.

### (5) Sensor Selectivity of “Locked” or “Open” P Strand

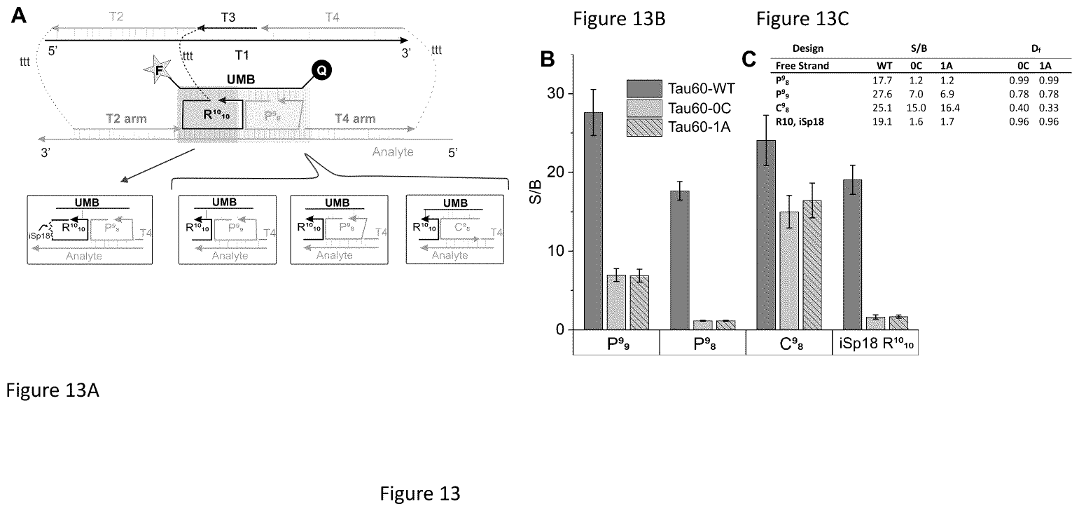

Since it was hypothesized that the capability of OWL structure for SNV differentiation is at least in part the consequence of the conformational strain of the small P and/or R strands folded in a circular form, the effect of the locked or open ends of the P strand is tested for achieving SNV differentiation with P strands P⁹₈ and P⁹₉, both having “locked” ends, and P strand C⁹₈ having open ends (5′-terminal nucleotides form base pairs with nucleotides on the oligonucleotide of UMB probe and 3′-terminal nucleotides with nucleotides on the analyte) (FIG. 4, FIG. 13). It was found that fluorescence of C⁹₈-equipped OWL2 sensor was higher than that of P⁹₈-equipped OWL2 sensor with Tau60-WT and Tau60 SNVs, showing poorer SNV differentiation (FIG. 4B, Table 1). Furthermore, substitution of P⁹₈ with P⁹₉, which has one more nucleotide and thus slightly increased flexibility of the nucleotide strand, also diminished SNV differentiation and increased overall fluorescent response (FIG. 4B), supporting the hypothesis that increased flexibility in P strand reduced SNV differentiation on the analyte.

However, the increased flexibility of R-strand by introducing a flexible iSp18 spacer between the UMB-binding and analyte-binding regions of R¹⁰₁₀ showed little effects on the selectivity and performance of OWL structure (FIG. 13).

### (6) Destabilizing Effect of a Single Nucleotide Gap Between T4 Arm and P Strand on the OWL Structure

Next, the effect of a gap between the P-strand and T4-arm on the function of OWL2 sensor was tested by introducing a gap.

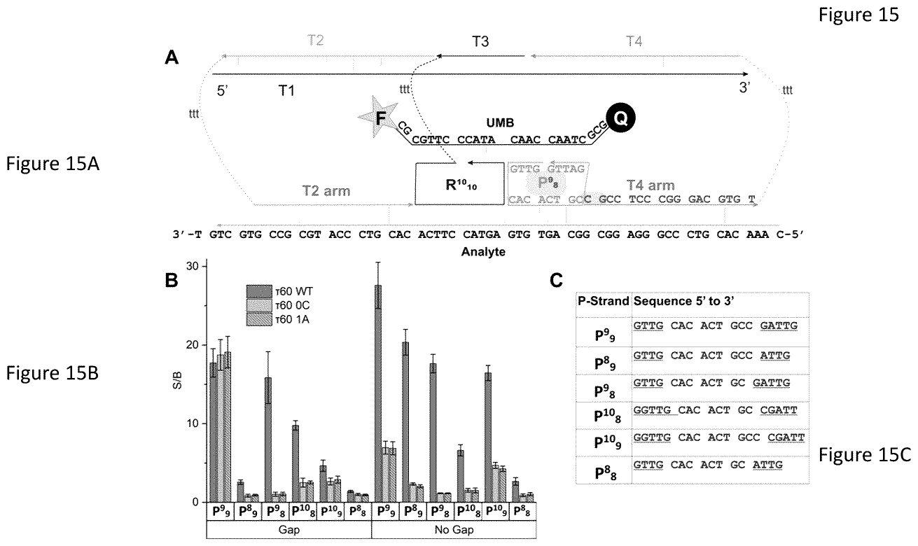

Although OWL2 sensor having P⁹₈ strand showed that the presence of a gap caused just slightly reduced fluorescence (S/B) without influence on SNV differentiation (FIG. 12B), overall, the presence of a gap between the P-strand and T4-arm reduced fluorescence (S/B) and SNV differentiation for all P stands (FIG. 15, Table 5). In the case where the relative location of the P strand and R strand are switched, the same no gap preference may be applied to T2-arm and P strand.

### (7) Redesigning of OWL2 Sensor for Another Analyte in a Cost-Efficient Manner

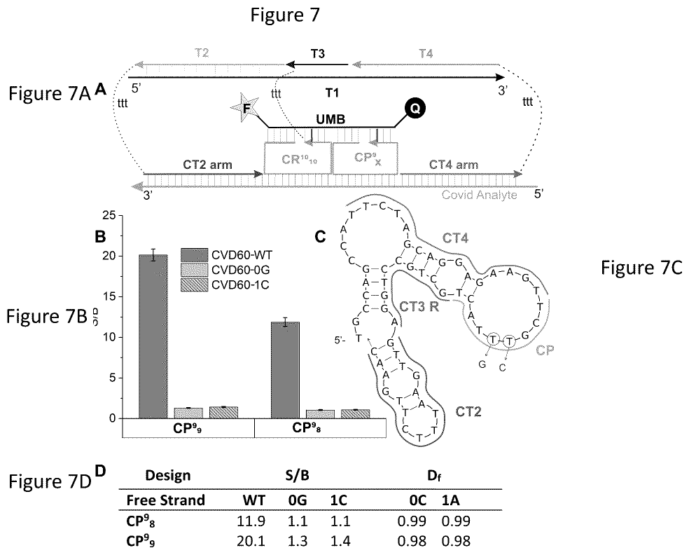

To ensure that OWL2 sensor can be used for detection of various SNVs on various analytes, OWL2 sensor was redesigned for a new sequence of another analyte, Covid-19 virus (FIG. 7C). Using the changed analyte-binding nucleotide sequences, including P-strand (named CP-strand for Covid-19) CP⁹₈ and CP⁹₉, Covid 19-WT and two SNVs were differentially detected at room temperature with excellent Dt of 0.98 and 0.99 (FIG. 7D).

In this embodiment, CP⁹₉-equipped OWL-2 sensor provided greater fluorescence (S/B) than CP⁹₈-equipped OWL-2 sensor (FIGS. 7B & D). This difference between Tau60-WT detection and Covid 19-WT detection with OWL2 sensor can be explained by the fact that Covid 19-WT's region complementary to P strand was A/T rich, whereas Tau60-WT's region complementary to P strand was relatively G/C rich. Thus, the effect of a SNV in the CVD60 analyte may be enough to destabilize the hybridization between the A/T rich 9 nucleotide-strand of the analyte binding region of CP⁹₉ strand/analyte without the need for the additional conformational strain inherent to CP⁹₈ strand having only 8 nucleotide-strand for the analyte SNV binding. Even though P⁹₈ provides excellent SNV differentiation for both analytes, it can be concluded that P⁹₉ design can also be used for A/T rich analytes. Overall, these results show that the OWL2 sensor design can be easily adapted for detection of any arbitrary analytes.

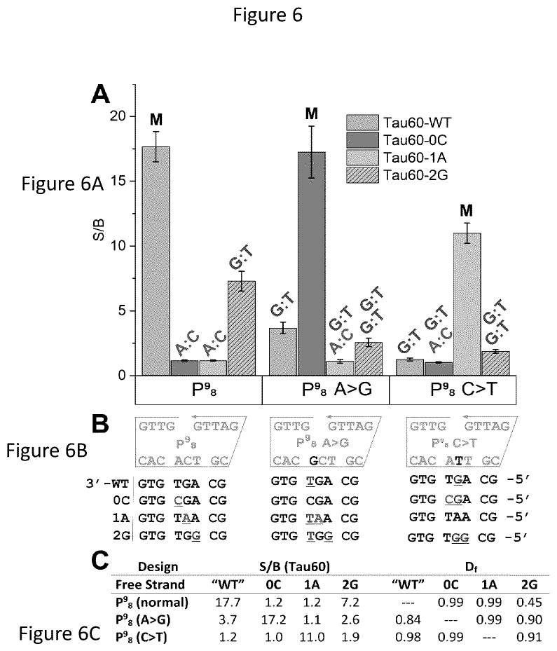

In addition, it was shown that OWL2 sensor can differentiate an analyte which forms a single G-T mismatch at room temperature (FIG. 6).

Based on the results described above, this disclosure provides an OWL2 sensor system as well as its use-kit and method for detecting at least one single nucleotide variation (SNV) in an analyte.

In one embodiment, a universal molecule beacon (UMB)-based hybridization sensor system is provided. The system includes
(a) an oligonucleotide R strand comprising: (i) at each of a 5′-terminus and 3′-terminus thereof, a region having a nucleotide sequence of 4-6 nucleotides, preferably a total of 10 nucleotides, complementary to a nucleotide sequence of a first region of an oligonucleotide of a UMB probe; and (ii) between the 5′-terminus and 3′-terminus thereof, a region having a nucleotide sequence of 8-12 nucleotides, preferably 10 nucleotides, complementary to a nucleotide sequence of a first region of an analyte;
(b) an oligonucleotide P strand comprising: (i) at each of a 5′-terminus and 3′-terminus thereof, a region having a nucleotide sequence of 4-6 nucleotides, preferably a total of 9 nucleotides, complementary to a nucleotide sequence of a second region of the oligonucleotide of the UMB probe; and (ii) between the 5′-terminus and 3′-terminus thereof, a region having a nucleotide sequence of 8-12 nucleotides, preferably 8-9 nucleotides, complementary to a nucleotide sequence of a second region of an analyte, which has at least one SNV;
wherein the R strand binding region on the analyte can be either 3′ location or 5′ location relative to the P strand binding region on the analyte, and wherein the R and P strand binding regions on the analyte are in proximity to each other;
(c) a UMB probe comprising (i) an oligonucleotide comprising a first region having a nucleotide sequence complementary to the 5′-terminus and 3′-terminus of the R strand, and a second region having a nucleotide sequence complementary to the 5′-terminus and 3′-terminus of the P strand; and comprising (ii) a fluorophore at one end of the oligonucleotide and a quencher at the other end of the oligonucleotide,
wherein the MB probe has a hairpin structure in the absence of an analyte, whereby the fluorophore and quencher interact in the absence of an analyte to quench fluorescence generated by the fluorophore; and
(d) a DNA scaffold comprising oligonucleotides of T2, T3, T4, and T1 strands, wherein the 5′-T1 strand-3′ hybridizes to the 5′-T4 strand-T3 strand-T2 strand-3′, and wherein the 5′-to-3′ direction of the T1 strand and the analyte is antiparallel to each other.

The T2 strand comprises an oligonucleotide arm (T2 arm) on the 3′-end of the T2 strand, and the T2 arm comprises 10-30 nucleotides that hybridize to a third region on the analyte at a 3′ location relative to the R and P strand binding regions on the analyte, and the T4 strand comprises an oligonucleotide arm (T4 arm) on the 5′-end of the T4 strand, wherein the T4 arm comprises 10-30 nucleotides that hybridize to a fourth region on the analyte at a 5′ location relative to the R and P strand binding regions on the analyte.

If the R strand binding region on the analyte is at a 3′ location relative to the P strand binding region on the analyte, the T2 arm binds to the third region on the analyte at a 3′ location relative to the R strand, and the T4 arm binds to the fourth region on the analyte at a 5′ location relative to the P strand; or if the R strand binding region on the analyte is at a 5′ location relative to the P strand binding region on the analyte, the T2 arm binds to the third region on the analyte at a 3′ location relative to the P strand, and the T4 arm binds to the fourth region on the analyte at a 5′ location relative to the R strand. T3 strand is connected at its 3′-terminus to the 5′ terminus of the R strand, or at its 5′-terminus to the 3′ terminus of the R strand. The R and P strands, UMB probe oligonucleotide, and an analyte form a four-stranded complex, and wherein the four-stranded complex is associated with the DNA scaffold of the T1 strand hybridized to T2, T3, T4 strands, via the T2 and T4 arms binding to the analyte and T3 strand linked to the R strand. Further, in the UMB-based hybridization sensor described above, there is no nucleotide gap on the analyte between the P strand binding region and T4 arm binding region if the R strand binding region on the analyte is 3′ location relative to the P strand binding region on the analyte; or between the P strand binding region and T2 arm binding region if the R strand binding region on the analyte is 5′ location relative to the P strand binding region on the analyte.

In another embodiment, a kit comprising the UMB-based hybridization sensor system described above and a plurality of oligonucleotide sets of T2-T2 arm, T3-R strand, T4-T4 arm, and P strand is provided, wherein the analyte binding regions of the R strand, P strand, T2 arm, and T4 arm are independently selected for specific bindings to various SNVs on various analytes.

In another embodiment, a method of identifying a single nucleotide variation in an analyte using the UMB-based hybridization sensor system describe above is provided, comprising the steps of:
(i) combining the oligonucleotides of T1, T2-T2 arm, T3-R strand, T4-T4 arm, the UMB probe, the P strand and an analyte in hybridization buffer, wherein the P strand is added after mixing the other components;
(ii) subjecting the mixture of (i) to conditions to permit annealing;
(iii) illuminating the second mixture at an excitation wavelength suitable for inducing fluorescence emission by the fluorophore; and
(iv) detecting the fluorescence emitted by the fluorophore, thereby detecting the presence of a single nucleotide variation in the analyte in the test sample.

### DNA Scaffold

### EXAMPLES

### Example 1. Materials and Methods

Reagents. All solutions were made using DNAse/protease-free water purchased from Fisher Scientific. Synthesized oligonucleotides were obtained from Integrated DNA Technologies, Inc (Coralville, IA) and concentrations of oligonucleotide stock solutions were quantified via absorbance at 260 nm on a Thermo Scientific NanoDrop One (Waltham, MA).

Annealing of DNA scaffolds. All tiles were annealed overnight (˜8-10 h) in 2 L of water after being heated to 95° C. and boiled for 5 min. Oligonucleotides were combined in a total volume of 1 mL with a concentration of 100 nM. Unless otherwise specified, tiles were annealed with variations of T1, T2, T3, and T4, but not the P-strand in hybridization buffer 1:50 mM Tris-HCl, 50 mM MgCl₂, 0.1% Tween-20, and pH 7.4.

Melt Curve Fluorescence Assays. Using DNA scaffolds previously annealed, UMB and ROX were added to the solutions to a final concentration of 25 nM, and P-strand was added to a final concentration of 200 nM. Bringing the total volume to 30 μL each using either water or analyte, the samples were then added to the 96-well plate. A control consisting of only UMB and ROX were used, in addition to a sample containing the DNA scaffold with no P-strand added, and a sample with no analyte added. After adding the samples to the plate, an optical adhesive cover was securely fitted to the top of the plate, and wells were sealed using a tool provided with the QuantStudio™ 6 Flex System. The plate was lightly flicked to eliminate bubbles and was vortexed and centrifuged for 20 s on a Fisher Scientific Mini Plate Spinner Centrifuge (Hamptom, NH). After allowing 30 min for annealing the plate was then placed into the QuantStudio™ Flex 6 system and cooled to 5° C. where it was held for 5 min. The fluorescence of the samples was then read continuously as the samples were heated from 5° C. to 70° C. (0.1° C./s). ROX was selected as a passive reference and FAM™ was read as the ‘Target’. Although the system was calibrated to account for well factors, background, and dye fluorescence, there were small variations between the background fluorescence of UMB and controls without analyte; therefore, there may be small fluorescence value variations observed depending on the date of the experiment. Data was exported to excel and subsequently to OriginLab 2021 (Northampton, MA) for data normalization and processing. The processed readings from at least three wells were averaged and plotted as a function of F_{FAM}/F_{ROX}. The derivative of fluorescence vs time was calculated by the QuantStudio™ Real-Time PCR Software to determine the melting temperature (Tₘ).

Limit of Detection. The limit of detection was determined for each study by conducting fluorescence experiments using a 60 μL quartz cuvette in a Perkin-Elmer (San Jose, CA) LS-55 Fluorescence Spectrophotometer with a xenon lamp. (λₗₑₓ=485 nm, λₑₘ=517 nm). The samples were used directly from annealed tiles and, to this solution, P-strand was added such that the final concentration was 200 nM and UMB was added to a final concentration of 25 nM. After the addition of analyte at varying concentrations, the samples were incubated in a 24° C. water bath for 30 min before being taken out of the bath and analyzed. Fluorescent values at 517 nm were recorded for three independent trials for each sample. The averages and standard deviations were plotted in Excel and OriginLab 2021 (Northampton, MA), and the linear region was found and fitted with an equation. The LOD was determined by using the equation with the fluorescent signal of the blank+3×Standard deviation of the blank.

Differentiation Fluorescent Assays. The differentiation of each tested sensor was determined by conducting fluorescence experiments in a similar manner to the limit of detection. For the OWL1 design, the samples were made such that Rₓ and P_{y} were added to final concentrations of 150 nM and 200 nM, respectively, unless stated otherwise. The samples were incubated in hybridization buffer 1 at room temperature (24° C.) for 20 min followed by measuring fluorescence intensity at 517 nm. The differentiation factor (Fₘ−F₀)/(Fₘₘ−F₀) was calculated by taking the Fluorescence of the Wild-Type analyte (Fₘ), subtracting the blank (F₀), and dividing it by the fluorescence of the respective SNV-containing analyte corrected by the blank. Differentiation was calculated using the fluorescent average of at least three trials.

Kinetics Assays. Using the same experimental conditions as for the other fluorescent assays, the fluorescence was measured over 45 minutes on a Cary Agilent Fluorimeter for 45 min. The OWL2 sensor and P-strand were mixed, analyte was added, and fluorescence was read immediately afterwards.

### Example 2. OWL2 Design and Performance

To overcome the limitations of the OWL1 sensor, OWL2 sensor was designed (FIG. 2). It also uses the UMB probe and P strand, but the free R strand of OWL1 was replaced with an association of DNA strands T1, T2, T3, and T4. The R strand was attached via a trithymidine linker to a fragment complementary to T1. Strands T2 and T4 contained long analyte binding arms, and T1 provided scaffolding for the complex formation. Together with R strand, the arms of T2 and T4 hybridized to the folded analyte and opened its secondary structure. The association of R, T2, and T4 with the analyte did not result in fluorescent signalling unless the P strand selectively hybridized with the SNV-containing site of the analyte and completed the OWL structure by allowing for the binding and subsequent opening of UMB.

As a model analyte for initial optimization of the OWL2 sensor, SNV ‘0C’ and ‘1A’ (FIG. 3A and Table 4†) found in the tau gene were chosen. These SNVs can lead to an increase in alternative splicing of exon 10, skewing the ratios of tau protein isoforms and causing Alzheimer's Disease (AD).^{35,36}

The secondary structures of synthetic fully matched analytes Tau60-WT and Tau18-WT are shown in FIG. 3A. The total energy of folded Tau60-WT is −11.34 kcal mol⁻¹, with the SNV-containing stem contributing to much of the stabilization.³⁶ It is important to note that the MB probe designed against Tau analyte failed in producing a fluorescent output.³⁷ Tau18-WT was designed to be fully complementary to strands P and R, but lacked the T2 and T4 binding sites. This short oligonucleotide formed a weak stem-loop structure, thus resembling a linear analyte under experimental conditions (FIG. 3A) and was used to study the effect of T2 and T4 arms on the sensor's performance.

The binding site of the P strand was chosen such that the two SNV sites corresponded to the middle positions of the strand for best SNV differentiation.³³ The analyte-binding site for the R strand, adjacent to the P strand binding site, formed a 10 base-pair (bp) duplex with the analyte and enabled both UMB-binding arms of the R strand to be positioned on the same side of the B DNA helix as needed for the formation of OWL structure.33 The T2- and T4-arms were chosen to have melting temperatures above the assay temperature (24° C.) and to have little or no secondary structures to ensure tight analyte association with the OWL nanostructure.

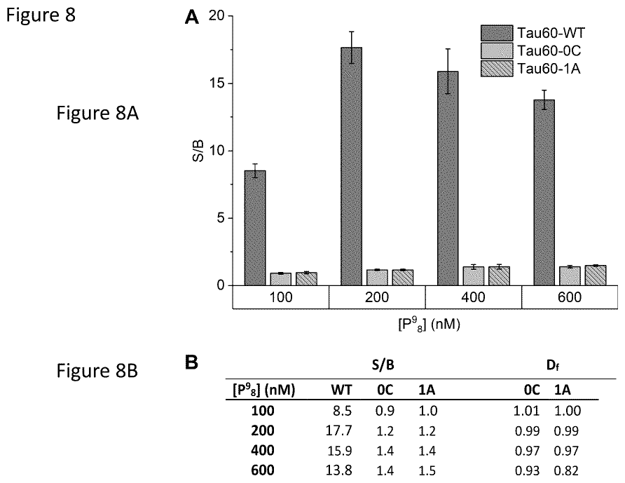

We optimized the concentration and sequences of the P strand to produce the highest signal-to-background ratio (S/B) and the greatest SNV differentiation (see details below). The optimal P strand had 9 nts and 8 nts complementary to the UMB probe and the analyte, respectively, and was, therefore named P⁹₈. It was used at the concentration of 200 nM, which provided the highest S/B (FIG. 8†). The optimized OWL2 sensor (FIG. 9C†) produced a S/B of ˜18 and maintained excellent selectivity that the OWL1 sensor exhibited for unstructured analytes (FIG. 3B). It was able to differentiate Tau60-WT from single-base mismatched Tau60-1A and Tau60-0C in the temperature range of 5-38° C. (FIG. 10†). This range is shifted toward low temperatures and almost 2 times broader than that for a typical MB probe that differentiates analytes with single base difference in the range of e.g. (53-70° C.).²⁶ The LOD for the folded Tau60-WT using OWL2 sensor was ˜0.4 nM (FIG. 12†), which was lower than that of the short Tau18-WT with OWL1 sensor (˜1.3 nM, FIG. 9B†), and falls in the range of LODs demonstrated by the best MB probes in detecting unfolded analytes.²⁸ To the best of our knowledge, this combination of high S/B and excellent selectivity in detecting folded analytes (FIG. 3B, 1st group of bars) is unprecedented.

Next, it was demonstrated that each feature of the OWL2 sensor contributes to at least one of the following functions: (1) enabling detection of folded analytes and (2) accurate discrimination of SNVs, (3) maintaining detection efficiency and selectivity over a range of ambient and low temperatures, and (4) ensuring low reagent cost due to “universality” of the UMB probe.

To overcome the limitations of OWL1 sensor, OWL2 sensor was designed (FIG. 1C). It uses a UMB probe and P strand. The R strand was replaced with the association of DNA strands T1, T2, T3, and T4. Strand R was covalently linked to T3. Strands T2 and T4 were equipped with additional analyte binding arms, which, together with R strand, could hybridize to folded analyte and open its secondary structure. However, this binding would not result in fluorescent signalling unless stand P selectively binds the SNV-containing site and completes the OWL structure formation.

As a model analyte, SNVs named ‘0C’ and ‘1A’ (FIG. 2, Table 4) found in the tau gene were selected, which can lead to an increase in alternative splicing of exon 10, skewing the ratios of tau protein isoforms and causing Alzheimer's Disease (AD).^{14,15} The secondary structures of synthetic fully matched analytes Tau60-WT and Tau18-WT are shown in FIG. 2. Tau60-WT folds with the total energy-11.34 kcal/mol, with the major stabilization contributed by Stem1 containing SNV of interest. Tau18-WT was designed to have all the nucleotides complementary to stands P and R, but lacking T2 and T4 arm binding sites. This short oligonucleotide formed almost no secondary structure under the experimental conditions (FIG. 2B) and was used to study the effect of T2 and T4 arms on sensor performance.

The binding site of P strand was chosen such that the two SNV sites were in the middle positions of the strand for best SNV differentiation.^{17,18} Binding site for R strand located next to P strand to form 10 bp duplex with the analyte. The 10 bp binding site satisfies the requirement for both UMB binding arm to face the same side of the analyte/strand R double stranded hybrid to allow UMB binding.¹³ T2 and T4 arms were chosen to have melting temperatures above the assay temperature (24° C.) and to have little or no secondary structures to ensure tight analyte association with the OWL tile.

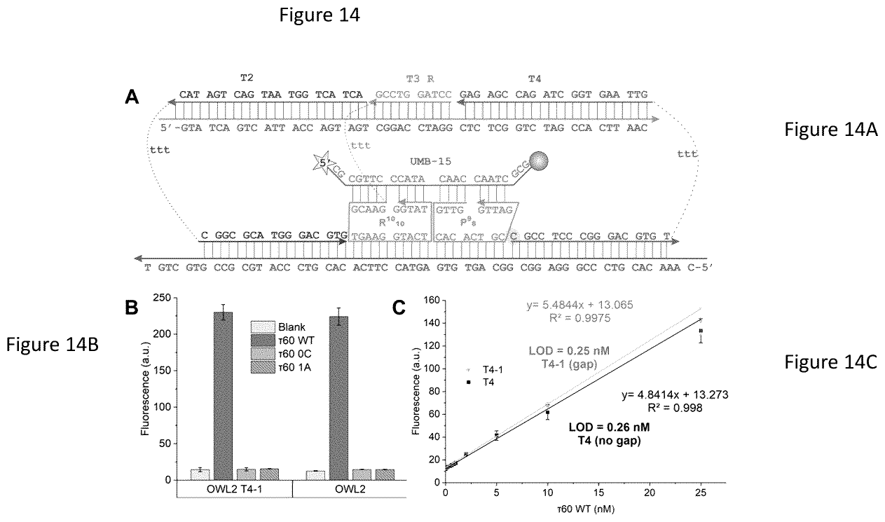

The concentrations of UMB probe and P strand (data not shown) were optimized, followed by optimization of the sequence of P strand to produce the highest signal-to-background ratio (S/B) and the greatest SNV differentiation (see details below). The optimal P strand had 8 nucleotides complementary to the analyte and two arms totaling 9 nucleotides complementary to UMB probe thus named P⁹₈. The optimized OWL2 sensor (FIG. 8) produced S/B of ˜18 and maintained the excellent selectivity of the OWL1 sensor (FIG. 2B). It was able to differentiate Tau60-WT from single base mismatched Tau60-1A, and -0C in the temperature range of 5-38° C. (FIG. 10). This range was ˜6° C. broader than that reported earlier for OWL1 sensor.¹⁰ The differentiation factors (D_{f}) were 1.0 and 0.99 for Tau60-0C and 1A, respectively (FIG. 12D, Table 5), where D_{f}=1.0 reflects the greatest possible selectivity: single base mismatched analyte produces only the background fluorescence. The LOD of OWL2 sensor was ˜0.25 nM (FIG. 14), which was one order of magnitude greater than that of OW1 sensor (˜5 nM),¹³ and falls in the range of LOD demonstrated by MB probes.⁷

Interesting to note that Tau18-WT lacking the fragments for binding T2 and T4 arms was detected with S/B ˜8 times lower than Tau60-WT. This suggests that the important function of T2 and T4 is not only in removing the structural constrain created by the Tau60-WT secondary structure, but also in localizing the analyte next to R strand, which overall stabilizes the OWL structure. On the other hand, OWL1 produced greater S/B than OWL2 sensor in complex with Tau18-WT (FIG. 2B). This can be explained by the reduced attraction of Tau18-WT to the bulky OWL2 structure due to the electrostatic repulsion. At the same time OWL1 sensor expectedly failed in detecting folded Tau60-WT analyte.

### Example 3. T2 and T4 Arms are Needed to Detect Folded Tau60-WT Analyte

The removal of the T2 and T4 arms resulted in a loss of the OWL2 ability to detect folded Tau60-WT (FIG. 3B, bars grouped as “OWL2 no arms”), which mimics the sensing capabilities of OWL1 (FIG. 3B, bars grouped as “OWL2 no arms”). The inclusion of the T2 and T4 arms decreases the energy barrier for hybridization to folded DNA sequences and allows for the opening of their secondary structures. Interestingly, OWL1 produced a lower signal with Tau18-WT than OWL2 in the presence of Tau60-WT (FIG. 3B). This suggests that an important function of T2 and T4 arms is not only to remove the structural constraint in the Tau60-WT structure, but also to position the analyte next to the R strand for tighter binding. Therefore, T2 and T4 arms are likely to participate in the stabilization of the OWL structure by increasing the local analyte concentration in proximity to the R strand.

On the other hand, OWL1 in complex with Tau18-WT produced a greater S/B than OWL2 lacking sensor T2 and T4 (“OWL2 no arms”) (FIG. 3B). This can be explained by the reduced attraction of Tau18-WT to the bulky OWL2 nanostructure due to electrostatic repulsion. At the same time, the OWL1 sensor expectedly failed in detecting the folded Tau60-WT analyte (FIG. 3B). Therefore, it could be concluded that the T2- and T4-arms are necessary for the detection of analytes folded in stable secondary structures.

### Example 4. Flexible Linkers Between R Stand and the DNA Scaffold Enable Higher S/B DNA Scaffold

Positioning of the fragile OWL structure near a bulky DNA scaffold formed by T1, T2, T3 and T4 in the OWL2 sensor might be challenging due to steric hindrance, which is hard to predict without knowing the crystal structure of the OWL2 sensor. The nature of the linker between the R strand and the scaffold-forming fragment of the T3 strand was varied ranging from the least flexible regular phosphodiester (PDE) to more flexible tri-thymidine (ttt), and the most flexible hexaethylene glycol (iSp18) linker (FIGS. 11 and 12†). For experiments with P⁹₉, it was found that an increase in linker flexibility resulted in a mild increase in fluorescence for both mismatched and matched analytes (FIG. 11†). In the case of P⁹₈, increased flexibility of a linker allowed for an increase in S/B for the fully matched analyte from ˜14 (PDE) to ˜18 (ttt) and to ˜25 (iSp18) without compromising the selectivity (FIG. 12†). Therefore, the S/B reported above for the optimal sensor can be increased from 18 to 25 by replacing ttt linker with iSp18 linker. This indicates that a spatial separation of the R strand from the scaffold is important for the stability of the OWL structure. The increase in S/B did not, however, change the LOD of the sensor (FIG. 12C†). In this work, therefore, it was considered that the increase of S/B for the iSp18 linker a minor advantage in comparison with the lower cost of the ttt linkers and conducted most of the experiments using the ttt linker equipped OWL2 sensor.

### Example 5

### Structural Constraint in the OWL Structure Promotes High Selectivity of the OWL Sensor

Following from our previous results,³³ it was hypothesized that the unprecedented SNV differentiation, at least in part, is the consequence of the conformational strain by the OWL structure and the locked ends (FIG. 1B) of the P strand.

First, the optimal P⁹₈ strand was redesigned to have opened ends, named C⁹₈ strand (FIG. 4A and Table 3†). Like all known probes, except OWL sensor, C⁹₈ strand had 5′ and 3′ ends unlocked: they were free to acquire any position relative to each other. It was found that fluorescence of C⁹₈-containing OWL2 was higher than that of P⁹₈-equipped OWL2 sensor. However, the sensor lost its selectivity (FIG. 4B, Table 1). Furthermore, substitution of P⁹₈ with P⁹₉ also diminished SNV differentiation and increased overall fluorescent response (FIG. 4B). observed increase in fluorescence can be explained by greater flexibility of the C⁹₈ and P⁹₉-equipped OWL2 sensor.

Next, the effect of the flexibility of R-strand on the selectivity and S/B was tested. For this purpose, an iSp18 spacer was introduced between the UMB-hybridizing and analyte-hybridizing regions of R¹⁰₁₀ near its 5′-end (FIG. 13A†). This flexible R¹⁰₁₀ strand was used with P⁹₈, and it was found that the S/B changed insignificantly with a noticeable reduction in SNV differentiation (FIG. 13B† and Table 1). Indeed, the differentiation factor (Df,³² Table 1) decreased from 0.99, which corresponds to a 100-fold higher fluorescent signal of the matched analyte being than that of the mismatch, to 0.96 (25-fold ration between the signals triggered by the matched and mismatched analytes). This data suggests the structural constrain of the R strand has lower effect on OWL sensor performance than the constraint contributed by the P strand.

Indeed, the constrained and rigid nature of the SNV-selective P-strand contributes the most to differentiation of WT from the mutants. By designing the P-strand with locked 5′- and 3′-ends in complex with UMB, a conformational strain was created that is unable to remain stable unless all 8 base pairs are complementary to the analyte. In the presence of a mis match, the strain experienced by P⁹₈ is great enough to inhibit P-strand hybridization to the analyte, which decomposes the OWL complex. If there are no mismatches, the P-strand is stabilized by the 8 base pairs complementary to the analyte, the stress of the conformational strain is insufficient to cause dissociation of the P-strand, and the scaffolding for UMB hybridization is complete.

TABLE 1
Signal to background ratio (S/B) and differentiation factor
(Df) for the OWL2 sensors containing three variations of the P strand.
Df = 1 − ΔFₘₘ/ΔFₘ, where ΔF represents
the signal of matched (m) or mismatched (mm) analyte with
the signal of the blank (no analyte)subtracted³²

| Design | |
|---|---|
| S/B | D_{f} |

| Free Strand | WT | 0C | 1A | 0C | 1A |
|---|---|---|---|---|---|
| P⁹₈ | 17.7 | 1.2 | 1.2 | 0.99 | 0.99 |
| P⁹₉ | 27.6 | 7.0 | 6.9 | 0.78 | 0.78 |
| C⁹₈ | 25.1 | 15.0 | 16.4 | 0.40 | 0.33 |
| R10, iSp18 | 19.1 | 1.6 | 1.7 | 0.96 | 0.96 |

### Example 6. Gap Effect and P-Strand Optimization

Previous studies have shown that the stability of multistranded DNA complexes are affected by the distance between adjacent DNA strands hybridized to a complementary nucleic acid.³⁸⁻⁴⁰ Therefore, a single nucleotide gap was introduced between the P-strand and T4-arm. The introduced gap did not significantly affect the S/B or DF of the optimal sensor containing P⁹₈ (FIGS. 12 and 14†). This indicates that the stability of the OWL structure does not depend on the staking interaction with the flanking T4 arm. However, a loss in the S/B or selectivity for the OWL2 sensor equipped with other P stands was noticed (FIG. 15 and Table 5†). Some of these undesired effects were explained by interaction of the P strand with the gap-forming nucleotide of the analyte. Therefore, it was concluded that OWL2 without a gap between P and T4 arm is preferable.

### Example 7. Detection of WT Analyte in the Presence of Mismatched Analyte

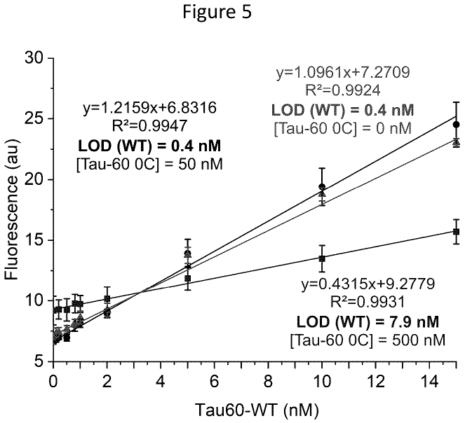

It was interesting to investigate if excellent selectivity of the OWL2 sensor allows detecting the matched analyte in the presence of excess amounts of a mismatched analyte. This capability of the sensor would be useful for detecting small fractions of cancerous DNA in an excess amount of healthy DNA for early-stage cancer diagnosis.²² The LOD of the fully matched Tau60-WT analyte was measured with the optimal OWL2 sensor in the presence of 50 nM Tau60-0C as a buffer component (FIG. 5). The LOD was found to be 0.4 nM, the same as in the absence of the mismatched analyte. This result indicated that the OWL2 sensor can differentiate from single-base mismatches and detect the fully matched analyte even when it makes up only 0.8% of the total analyte, which is comparable with the state-of-the-art fluorescent sensors.^{22,41} An increase of the mismatched Tau60-0C analyte to 500 nM required an increase in the OWL2 (T1/T2/T3/T4) to 600 nM and a decrease in P⁹₈ to 50 nM in order to offset some of the background fluorescence. It was found that the concentration of analytes should not exceed OWL2 (T1/T2/T3/T4 association) sensor concentration, likely due to the hybridization of T2- and T4-arms to analyte, even when it contains a mismatch. Due to high OWL2 concentration, the background fluorescence was high, which resulted to high LOD of ˜8 nM (FIG. 5). Therefore, further sensor optimization is needed to improve the detection of low fractions of the true targets in the presence of single base mismatched analytes.

### Example 8. G:T Discrimination

G-T mismatches are known to be the least destabilizing of all base-mispairing scenarios and, therefore, the most challenging to discriminate.^{42,43} Here, it was investigated if the OWL2 sensor is capable of differentiating an analyte that forms a single G-T mismatch with the sensor. It was found that P⁹₈ has Df of 0.45 when tested against the Tau60-2G analyte, which has an A >G substitution (FIG. 6C and FIG. 16D† for structure). The effect of two other G-T mismatches was also tested by changing the sequence of the P-strand: P⁹₈ A >G and P⁹₈ C>T (FIG. 6B and Table 3†) had full complementarity to the Tau60-0C and Tau60-1A analytes, respectively. They were able to discriminate against G-T mismatches with a Df of 0.84 and 0.98. (FIG. 6).

Discrimination using the original P⁹₈ was expectedly poor since the G-T was situated between the two stable G-C base pars and shifted from the middle of the stand P-analyte hybrid. Mismatches on the ends of hybridization sites are known to be less destabilizing than those in the center. 32.33 Expectedly, the mismatches closer to the center (P⁹₈ A >G and P⁹₈ C >T) were better discriminated. However, P⁹₈ C >T had a greater A/T content, which possibly led to the best discrimination of the three. It was shown that, through modification of the P-strand, it was possible to differentiate even G-T mismatches, with the best discriminating ability of the sensors containing G-T mismatch in the middle position of stand P/analyte complex and when flanked by A-T base pairs (FIG. 6A, 3rd group of bars).

### Example 9. Detection of RNA Analyte

Since the characteristics of RNA/DNA helical structure are somewhat different due to the difference in ribose and deoxyribose conformation,⁴⁴ it was investigated if the same OWL-2 sensor that performs well with DNA analytes is suitable for detecting an RNA analyte. It was found that the OWL2 sensor equipped with P⁹₈ strand was able to detect Tau60-WT RNA at a LOD of 0.8 nM, which is comparable to the 0.4 nM LOD of Tau-60 DNA. The ability of the OWL sensor to detect RNA may have practical significance since Tau-60 DNA is associated with the development of Alzheimer's disease.^{35,36}

### Example 10. OWL2 Sensor can be Redesigned for Another Analyte in a Cost-Efficient Manner

To ensure that OWL2 sensor can be easily redesigned for other analytes, it was applied to a sequence from the Covid-19-causing SARS-CoV2 virus). By only changing the analyte-binding portions of T2, T3, T4, i.e., T2-arm, R strand, and T4-arm sequences, and P-strand (named CP-strand for Covid-19), it was possible to show that both the CP⁹₈ and CP⁹₉ allowed for differentiation of the fully matched CVD60-WT from the mismatched CVD60-1C and CVD60-0G analytes (FIG. 7). This was found to be juxtaposed with the Tau-specific OWL2 sensor, which was not specific when equipped with P⁹₉. This different sensor behaviour could be explained by the A/T-rich sequence complementary to CP⁹₉ in CVD60-WT. It was speculated that if the P-strand binding region is A/T rich, the P⁹₉ may still provide selectivity. However, this statement should be verified with other sets of analyte.

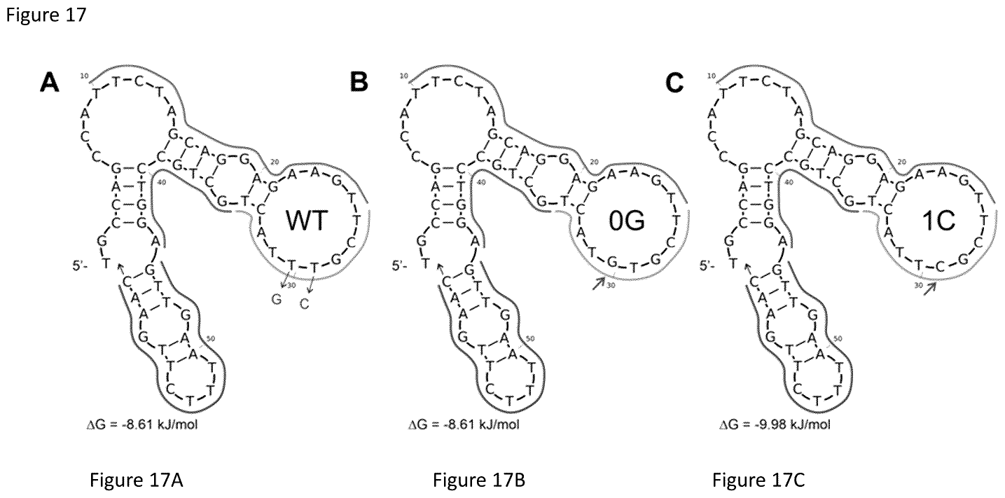

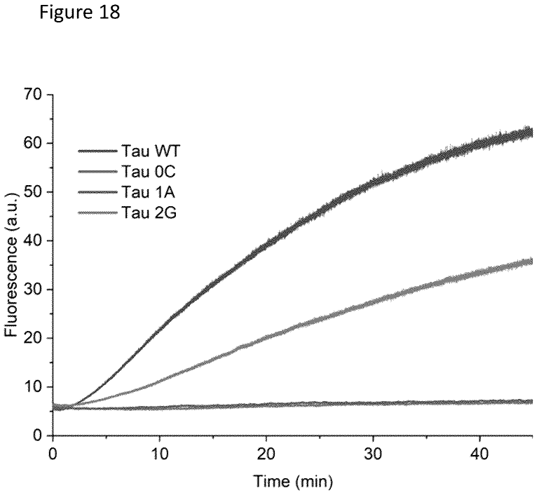

Overall, these results show that the OWL2 design can be easily adapted to detect another analyte without the need for costly changes. The cost of one nucleotide addition in IDT Inc. is $0.42 (minimum synthetic scale), which comes to 56.7 USD for adaptation of T2, T3, T4, and P-strand to each new analyte. At the same time, the cost of a new MB probe is ˜350 USD (minimum synthetic scale) due to the need for conjugation of the oligonucleotide with two dyes and double HPLC purification. Additionally, the design of an MB probe for each new analyte is known to be associated with many problems, such as stem invasion and loop interference, to the degree that it is impossible to design an efficient MB probe for some analytes.28,32 By designing the UMB-hybridizing regions of R- and P strands to be independent of the analyte sequence, it is allowed for the UMB technology to be applied to analytes of potentially any sequence. Furthermore, it was shown that OWL2 design is applicable to both DNA and RNA analytes which contain an SNV in both the stem and the loop regions (FIGS. 16 and 17†).

TABLE 2
Oligonucleotides used in the assembly of variations of OWL2 Sensor

| Name | Sequence 5'->3' |
|---|---|
| UMB | 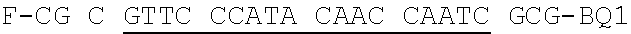 |
| (SEQ ID NO: 1) | |
| T1 | 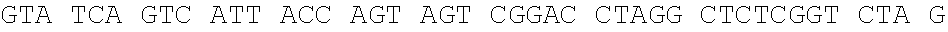 |
| (SEQ ID NO: 2) | 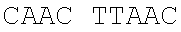 |
| T2₂ | ACT ACT GGT AAT GAC TGA TAC ttt C GGC GCA TGG GAC GTG |
| (SEQ ID NO: 3) | |
| T3₂-9 no linker | 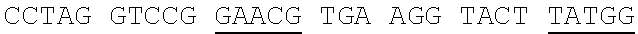 |
| (SEQ ID NO: 4) | |
| T3₂-9-ttt | 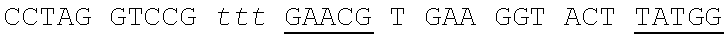 |
| (SEQ ID NO: 5) | |
| T3₂-9 iSp | 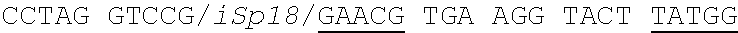 |
| (SEQ ID NO: 6 | |
| and 52) | |
| T4₂ | 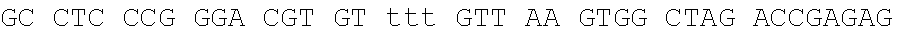 |
| (SEQ ID NO: 7) | |
| T3₂-9 + T4₂ + 1 | 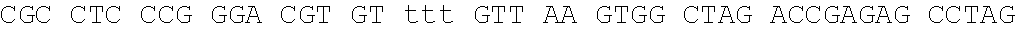 |
| (SEQ ID NO: 8) | 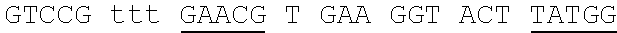 |
| T4₂ + 1 | 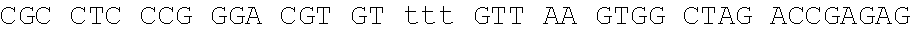 |
| (SEQ ID NO: 9) | |
| T4₂-1 | 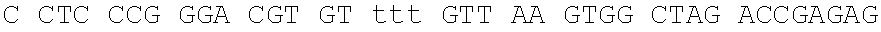 |
| (SEQ ID NO: 10) | |
| CT2 | ACT ACT GGT AAT GAC TGA TAC ttt GTTC AAGA AATT CAAC |
| (SEQ ID NO: 11) | |
| CT3-9 | 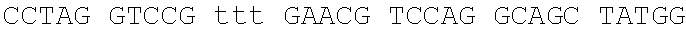 |
| (SEQ ID NO: 12) | |
| CT4 | 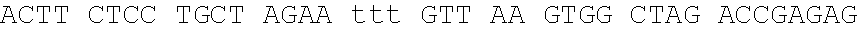 |
| (SEQ ID NO: 13) | |
| CT4 + 1 | 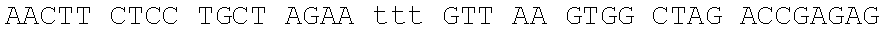 |
| (SEQ ID NO: 14) | |

F, fluorescein;
BQ1, black hole quencher 1;
underlined are the fragments complementary to UMB probe;
ttt, tri-thymidine linkers between tile-forming fragments and the analyte binding arms;
/iSp18/ internal spacer 18.

TABLE 3
Sequences of P-strand variations used

| Name | Sequence 5′→3′ |
|---|---|
| P⁹₉ | GTTG CAC ACT GCC GATTG |
| (SEQ ID NO: 15) | |
| P⁸₉ | GTTG CAC ACT GCC ATTG |
| (SEQ ID NO: 16) | |
| P⁹₈ | GTTG CAC ACT GC GATTG |
| (SEQ ID NO: 17) | |
| P¹⁰₈ | GGTTG CAC ACT GC CGATT |
| (SEQ ID NO: 18) | |
| P¹⁰₉ | GGTTG CAC ACT GCC CGATT |
| (SEQ ID NO: 19) | |
| P⁸₈ | GTTG CAC ACT GC ATTG |
| (SEQ ID NO: 20) | |
| P⁹₇ | GTTG CAC ACT G GATTG |
| (SEQ ID NO: 21) | |
| CP⁹₉ | GTTG AGTA AACGA GATTG |
| (SEQ ID NO: 22) | |
| CP⁹₈ | GTTG AGTA AACG GATTG |
| (SEQ ID NO: 23) | |

Underlined are the fragments complementary to UMB probe.

TABLE 4
Sequences of the analytes used in this study.

| Name | Sequence 5′→3′ |
|---|---|
| Tau60-WT | CA AAC ACG TCC CGG GAG GC G GCA GTG TGA GTA CCT TCA C AC GTC |
| (SEQ ID | CCA TGC GCC GTG CTG T |
| NO: 24) | |
| Tau60-0C | CA AAC ACG TCC CGG GAG GC G GCA GCG TGA GTA CCT TCA C AC GTC |
| (SEQ ID | CCA TGC GCC GTG CTG T |
| NO: 25) | |
| Tau60-1A | CA AAC ACG TCC CGG GAG GCG GCA ATG TGA GTA CCT TCA C AC GTC |
| (SEQ ID | CCA TGC GCC GTG CTG T |
| NO: 26) | |
| Tau60-2G | CA AAC ACG TCC CGG GAG GCG GCG GTG TGA GTA CCT TCA C AC GTC |
| (SEQ ID | CCA TGC GCC GTG CTG T |
| NO: 27) | |
| Tau19-WT | GCA GTG TGA GTA CCT TCA C |
| (SEQ ID | |
| NO: 28) | |
| Tau19-0C | GCA GCG TGA GTA CCT TCA C |
| (SEQ ID | |
| NO: 29) | |
| Tau19-1A | GCA ATG TGA GTA CCT TCA C |
| (SEQ ID | |
| NO: 30) | |
| Tau18_WT | GCA GTG TGA GTA CCT TCA |
| (SEQ ID | |
| NO: 31) | |
| Tau18_0C | GCA GCG TGA GTA CCT TCA |
| (SEQ ID | |
| NO: 32) | |
| Tau18_1A | GCA ATG TGA GTA CCT TCA |
| (SEQ ID | |
| NO: 33) | |
| CVD60_WT | TGC CAG CCA TTC TAG CAG GAG AAGT TCG TTT ACT GCT GCC TGG A G |
| (SEQ ID | TTG AAT TTC TTG AAC |
| NO: 34) | |
| CVD60_1C | TGC CAG CCA TTC TAG CAG GAG AAGT TCG CTT ACT GCT GCC TGG A G |
| (SEQ ID | TTG AAT TTC TTG AAC |
| NO: 35) | |
| CVD60_0G | TGC CAG CCA TTC TAG CAG GAG AAGT TCG TGT ACT GCT GCC TGG A G |
| (SEQ ID | TTG AAT TTC TTG AAC |
| NO: 36) | |

SNV sites are in red; underlined are the fragments complementary to P and R strands.

TABLE 5
Signal to background (S/B) and differentiation factor (D_{f}) for all
analytes with varying combinations of P-strand and Gap or No Gap.

| | | |
|---|---|---|
| Design | S/B | D_{f} |

| Gap | P-strand | WT | 0C | 1A | 0C | 1A |
|---|---|---|---|---|---|---|
| + | P⁹₉ | 17.7 | 18.8 | 19.1 | −0.06 | −0.08 |
| + | P⁸₉ | 2.6 | 0.9 | 0.9 | 1.09 | 1.05 |
| + | P⁹₈ | 15.9 | 1.0 | 1.1 | 1.00 | 0.99 |
| + | P¹⁰₈ | 9.8 | 2.5 | 2.5 | 0.83 | 0.82 |
| + | P¹⁰₉ | 4.7 | 2.7 | 2.9 | 0.55 | 0.48 |
| + | P⁸₈ | 1.4 | 1.0 | 0.95 | 1.06 | 1.06 |
| − | P⁹₉ | 27.6 | 7.0 | 6.9 | 0.78 | 0.78 |
| − | P⁸₉ | 20.4 | 2.3 | 2.0 | 0.93 | 0.95 |
| − | P⁹₈ | 17.7 | 1.2 | 1.2 | 0.99 | 0.99 |
| − | P¹⁰₈ | 6.6 | 1.5 | 1.5 | 0.90 | 0.91 |
| − | P¹⁰₉ | 16.5 | 4.7 | 4.3 | 0.76 | 0.79 |
| − | P⁸₈ | 2.7 | 0.9 | 1.1 | 1.06 | 0.96 |

### REFERENCES

1. J. C. e. a. Venter, Science, 2001, 291, 1304-1351.
2. R. Sachidanandam, D. Weissman, S. C. Schmidt, J. M. Kakol, L. D. Stein, G. Marth, S. Sherry, J. C. Mullikin, B. J. Mortimore and D. L. Willey, Nature, 2001, 409, 928-934.
3. J. Hanson, D. Brezavar, S. Hughes, S. Amudhavalli, E. Fleming, D. Zhou, J. T. Alaimo and P. E. Bonnen, Clin. Genet., 2022, 101, 214-220.
4. M. A. Field, Immunol. Cell Biol., 2021, 99, 146-156.
5. P. D. Stenson, E. V. Ball, M. Mort, A. D. Phillips, J. A. Shiel, N. S. T. Thomas, S. Abeysinghe, M. Krawczak and D. N. Cooper, Hum. Mutat., 2003, 21, 577-581.
6. S. H. Jiang, V. Athanasopoulos, J. I. Ellyard, A. Chuah, J. Cappello, A. Cook, S. B. Prabhu, J. Cardenas, J. Gu, M. Stanley, J. A. Roco, I. Papa, M. Yabas, G. D. Walters, G. Burgio, K. Mckeon, J. M. Byers, C. Burrin, A. Enders, L. A. Miosge, P. F. Canete, M. Jelusic, V. Tasic, A. C. Lungu, S. I. Alexander, A. R. Kitching, D. A. Fulcher, N. Shen, T. Arsov, P. A. Gatenby, J. J. Babon, D. F. Mallon, C. de Lucas Collantes, E. A. Stone, P. Wu, M. A. Field, T. D. Andrews, E. Cho, V. Pascual, M. C. Cook and C. G. Vinuesa, Nat. Commun., 2019, 10, 2201.
7. G. Cao, Y. Qiu, K. Long, Y. Ma, H. Luo, M. Yang, J. Hou, D. Huo and C. Hou, Anal. Chem., 2022, 94, 17653-17661.
8. C. Graham, A. Eshaghi, A. Sarabia, S. Zittermann, P. Stapleton, J. V. Kus and S. N. Patel, Access Microbiol., 2020, 2, acmi000111.
9. T.-L. Li, M.-W. Wu, W.-C. Lin, C.-H. Lai, Y.-H. Chang, L.-J. Su and W.-Y. Chen, Anal. Bioanal. Chem., 2019, 411, 3871-3880.
10. C. P. Paweletz, A. G. Sacher, C. K. Raymond, R. S. Alden, A. O'Connell, S. L. Mach, Y. Kuang, L. Gandhi, P. Kirschmeier, J. M. English, L. P. Lim, P. A. Janne and G. R. Oxnard, Clin. Cancer Res., 2016, 22, 915-922.
11. T. Li, H. Zou, J. Zhang, H. Ding, C. Li, X. Chen, Y. Li, W. Feng and K. Kageyama, Analyst, 2022, 147, 3993-3999.
12. M. Azhar, R. Phutela, M. Kumar, A. H. Ansari, R. Rauthan, S. Gulati, N. Sharma, D. Sinha, S. Sharma, S. Singh, S. Acharya, S. Sarkar, D. Paul, P. Kathpalia, M. Aich, P. Sehgal, G. Ranjan, R. C. Bhoyar, K. Singhal, H. Lad, P. K. Patra, G. Makharia, G. R. Chandak, B. Pesala, D. Chakraborty and S. Maiti, Biosens. Bioelectron., 2021,183, 113207.
13. V. Taly, D. Pekin, L. Benhaim, S. K. Kotsopoulos, D. LeCorre, X. Li, I. Atochin, D. R. Link, A. D. Griffiths, K. Pallier, H. Blons, O. Bouché, B. Landi, J. B. Hutchison and P. Laurent-Puig, Clin. Chem., 2013, 59, 1722-1731.
14. S. Bai, B. Xu, Y. Zhang, Y. Zhang, H. Dang, S. Yang, C. Zuo, L. Zhang, J. Li and G. Xie, Biosens. Bioelectron., 2020, 154, 112092.
15. N. Zhang and D. H. Appella, J. Infect. Dis., 2010, 201, S42-S45.
16. M. B. Thayer, J. M. Lade, D. Doherty, F. Xie, B. Basiri, O. S. Barnaby, N. S. Bala and B. M. Rock, Sci. Rep., 2019, 9, 3566.
17. F. Bekkaoui, I. Poisson, W. Crosby, L. Cloney and P. Duck, BioTechniques, 1996, 20, 240-248.
18. Q. Huang, Z. Liu, Y. Liao, X. Chen, Y. Zhang and Q. Li, PLOS One, 2011, 6, e19206.
19. S. Tyagi and F. R. Kramer, Nat. Biotechnol., 1996, 14, 303-308.
20. V. V. Demidov and M. D. Frank-Kamenetskii, Trends Biochem. Sci, 2004, 29, 62-71.
21. D. M. Kolpashchikov, Chem. Rev., 2010, 110, 4709-4723.
22. R. Van Hoof, M. Szymonik, S. K. Nomidis, K. Hollanders, A. Jacobs, I. Nelissen, P. Wagner and J. Hooyberghs, Sens. Actuators, B, 2022, 368, 132175.
23. L. Chen, H. Huang, Z. Wang, K. Deng and H. Huang, Talanta, 2022, 243, 123352.
24. X. Ke, Y. Ou, Y. Lin and T. Hu, Biosens. Bioelectron., 2022, 212, 114428.
25. M. Ahmed, N. M. Pollak, G. J. Devine and J. Macdonald, Sens. Actuators, B, 2022, 367, 132085.
26. M. Stancescu, T. A. Fedotova, J. Hooyberghs, A. Balaeff and D. M. Kolpashchikov, J. Am. Chem. Soc., 2016, 138, 13465-13468.
27. P. Hardinge and J. A. H. Murray, BMC Biotechnol., 2019, 19, 55.
28. D. M. Kolpashchikov, Scientifica, 2012, 2012.
29. E. Navarro, G. Serrano-Heras, M. J. Castaño and J. Solera, Clin. Chim. Acta, 2015, 439, 231-250.
30. S. A. E. Marras, S. Tyagi and F. R. Kramer, Clin. Chim. Acta, 2006, 363, 48-60.
31. M. W. McCarthy and T. J. Walsh, Expert Rev. Mol. Diagn., 2016, 16, 1025-1036.
32. C. Nguyen, J. Grimes, Y. V. Gerasimova and D. M. Kolpashchikov, Chemistry, 2011, 17, 13052-13058.
33. R. J. Karadeema, M. Stancescu, T. P. Steidl, S. C. Bertot and D. M. Kolpashchikov, Nanoscale, 2018, 10, 10116-10122.
34. J. N. Zadeh, C. D. Steenberg, J. S. Bois, B. R. Wolfe, M. B. Pierce, A. R. Khan, R. M. Dirks and N. A. Pierce, J. Comput. Chem., 2011, 32, 170-173.
35. M. Goedert and R. Jakes, Biochim. Biophys. Acta, 2005, 1739, 240-250.
36. M. Hasegawa, M. J. Smith, M. lijima, T. Tabira and M. Goedert, FEBS Lett., 1999, 443, 93-96.
37. J. Grimes, Y. V. Gerasimova and D. M. Kolpashchikov, Angew. Chem., Int. Ed., 2010, 49, 8950-8953.
38. S. Cai, C. Lau and J. Lu, Anal. Chem., 2010, 82, 7178-7184.
39. P. Yakovchuk, E. Protozanova and M. D. FrankKamenetskii, Nucleic Acids Res., 2006, 34, 564-574.
40. D. V. Pyshnyi, S. G. Lokhov, M. A. Podyminogin, E. M. Ivanova and V. F. Zarytova, Nucleosides, Nucleotides Nucleic Acids, 2000, 19, 1931-1941.
41. D. M. Kolpashchikov, J. Am. Chem. Soc., 2006, 128, 10625-10628.
42. F. Aboul-Ela, D. Koh, I. Tinoco Jr. and F. H. Martin, Nucleic Acids Res., 1985, 13, 4811-4824.
43. X. Piao, L. Sun, T. Zhang, Y. Gan and Y. Guan, Acta Biochim. Pol., 2008, 55, 713-720.
44. J. I. Gyi, A. N. Lane, G. L. Conn and T. Brown, Biochemistry, 1998, 37, 73-80.

What is claimed is:

1. A universal molecule beacon (UMB)-based hybridization sensor system for detecting at least one single nucleotide variation (SNV) in an analyte, comprising:
(a) an oligonucleotide R strand comprising: (i) at each of a 5′-terminus and 3′-terminus thereof, a region having a nucleotide sequence of 4-6 nucleotides, optionally a total of 10 nucleotides, complementary to a nucleotide sequence of a first region of an oligonucleotide of a UMB probe; and (ii) between the 5′-terminus and 3′-terminus thereof, a region having a nucleotide sequence of 8-12 nucleotides, optionally 10 nucleotides, complementary to a nucleotide sequence of a first region of an analyte;
(b) an oligonucleotide P strand comprising: (i) at each of a 5′-terminus and 3′-terminus thereof, a region having a nucleotide sequence of 4-6 nucleotides, optionally a total of 9 nucleotides, complementary to a nucleotide sequence of a second region of the oligonucleotide of the UMB probe; and (ii) between the 5′-terminus and 3′-terminus thereof, a region having a nucleotide sequence of 8-12 nucleotides, optionally 8-9 nucleotides, complementary to a nucleotide sequence of a second region of the analyte, which has at least one SNV;
wherein the R strand binding region on the analyte can be either 3′ location or 5′ location relative to the P strand binding region on the analyte, and wherein the R and P strand binding regions on the analyte are in proximity to each other;
(c) a UMB probe comprising (i) an oligonucleotide comprising a first region having a nucleotide sequence complementary to the 5′-terminus and 3′-terminus of the R strand, and a second region having a nucleotide sequence complementary to the 5′-terminus and 3′-terminus of the P strand; and comprising (ii) a fluorophore at one end of the oligonucleotide and a quencher at the other end of the oligonucleotide,
wherein the UMB probe has a hairpin structure in the absence of an analyte, whereby the fluorophore and quencher interact in the absence of an analyte to quench fluorescence generated by the fluorophore; and
(d) a DNA scaffold comprising T2, T3, T4, and T1 oligonucleotide strands,
wherein the 5′-T1 strand-3′ hybridizes to the 5′-T4 strand-T3 strand-T2 strand-3′, and wherein the 5′-to-3′ direction of the T1 strand and the analyte is antiparallel to each other;
wherein the T2 strand comprises an oligonucleotide arm (T2 arm) on the 3′ end of the T2 strand, the T2 arm comprising 10-30 nucleotides that hybridize to a third region on the analyte at a 3′ location relative to the R and P strand binding regions on the analyte;
wherein the T4 strand comprises an oligonucleotide arm (T4 arm) on the 5′ end of the T4 strand, the T4 arm comprising 10-30 nucleotides hybridizes to a fourth region on the analyte at a 5′ location relative to the R and P strand binding regions on the analyte,
wherein if the R strand binding region on the analyte is 3′ location relative to the P strand binding region on the analyte, the T2 arm binds to the third region on the analyte at a 3′ location relative to the R strand, and the T4 arm binds to the fourth region on the analyte at a 5′ location relative to the P strand;
wherein if the R strand binding region on the analyte is 5′ location relative to the P strand binding region on the analyte, the T2 arm binds to the third region on the analyte at a 3′ location relative to the P strand, and the T4 arm binds to the fourth region on the analyte at a 5′ location relative to the R strand
wherein the T3 strand is connected at its 3′-terminus to the 5′ terminus of the R strand, or at its 5′-terminus to the 3′ terminus of the R strand;
wherein the R and P strands, UMB probe oligonucleotide, and an analyte form a four-stranded complex, and wherein the four-stranded complex is associated with the DNA scaffold of the T1 strand hybridized to the T2 and T4 strands via the T2 and T4 arms hybridizing to the analyte and to the T3 strand via the R strand,

2. The UMB-based hybridization sensor of claim 1, wherein the T1 strand comprises a nucleotide sequence comprising SEQ ID NO:2.

3. The UMB-based hybridization sensor of claim 1, wherein any of T2, T3, T4 or T1 strands are biotinylated to attach the DNA scaffold to a streptavidin-coated substrate or beads.

4. The UMB-based hybridization sensor of claim 1, wherein the nucleotide sequence of the T2 strand comprises 5′-ACT ACT GGT AAT GAC TGA TAC-3′ (SEQ ID NO: 49) or SEQ ID NO: 3.

5. The UMB-based hybridization sensor of claim 1, wherein the nucleotide sequence of the T4 strand comprises 5′-GTT AA GTGG CTAG ACCGAGAG-3′ (SEQ ID NO: 51) or SEQ ID NO: 6.

6. The UMB-based hybridization sensor of claim 1, wherein the nucleotide sequences of the T2 arm and T4 arm are chosen to have melting temperatures above room temperature (24° C.) and to have little or no secondary structures to ensure tight analyte association with the DNA tile.

7. The UMB-based hybridization sensor of claim 1, wherein the nucleotide sequence of the T3 strand comprises 5′-CCTAG GTCCG-3′ (SEQ ID NO: 50), SEQ ID NO: 4, SEQ ID NO: 5, or SEQ ID NO; 6.

8. The UMB-based hybridization sensor of claim 1, wherein the T2 strand is connected to the T2 arm without or with at least one linker selected from tri-thymidine (ttt) or hexaethylene glycol.

9. The UMB-based hybridization sensor of claim 1, wherein the T4 strand is connected to the T4 arm without or with at least one linker selected from tri-thymidine (ttt) or hexaethylene glycol.

10. The UMB-based hybridization sensor of claim 1, wherein the T3 is connected to/comprises the R strand without or with 1 to 10 linkers selected from tri-thymidine or hexaethylene glycol.

11. The UMB-based hybridization sensor of claim 1, wherein the P strand has locked ends at its 5′-terminus and 3′-terminus.

12. The UMB-based hybridization sensor of claim 1, wherein there is no nucleotide gap on the analyte between the P strand binding region and T4 arm binding region if the R strand binding region on the analyte is 3′ location relative to the P strand binding region on the analyte; or between the P strand binding region and T2 arm binding region if the R strand binding region on the analyte is 5′ location relative to the P strand binding region on the analyte.

13. The UMB-based hybridization sensor of claim 1, further comprising a plurality of oligonucleotide sets of T2-T2 arm, T3-R strand, T4-T4 arm, and P strand, wherein the analyte binding regions of the R strand, P strand, T2 arm, and T4 arm are independently selected for specific bindings to one or more SNVs on one or more analytes.

14. The UMB-based hybridization sensor of claim 1, wherein the analyte is single stranded or double stranded DNA, single stranded or double stranded RNA, a DNA/RNA hybrid or a variant thereof.

15. The UMB-based hybridization sensor of claim 1, wherein the oligonucleotide of the UMB-probe is an oligonucleotide of SEQ ID NO:1.

16. The UMB-based hybridization sensor of claim 1, wherein the fluorophore can be a single fluorophore or combination of at least 2 fluorophores, selected from, but not intended to be limited to, fluorescein amidite (FAM), fluorescein isothiocyanate (FITC), 4,5,6,7-tetrachlorofluorescein, 6-carboxy-2′,4,4′,5′,7,7-hexachlorofluorescein, cyanine dyes Cy2, Cy3, Cy3.5, Cy5, Cy5.5 Cy7, Cy7.5 (ranging from green to near-infrared), Texas Red, rhodamine 123 (hydrochloride), sulforhodamine 101 acid chloride succinimidyl ester, 2-3-(dimethylamino)-6-dimethyliminio-xanthen-9-ylbenzoate, (2E)-2-(2E,4E)-5-(2-tert-butyl-9-ethyl-6,8,8-trimethyl-pyrano 3,2-gquinolin-1-ium-4-yl) penta-2,4-dienylidene-1-(6-hydroxy-6-oxo-hexyl)-3,3-dimethylindoline-5-sulfonate, and the like, and wherein the quencher is selected according to the emission range of the selected fluorophore.

17. A kit comprising the UMB-based hybridization sensor system of claim 1 for detecting at least one single nucleotide variation (SNV) in an analyte;
a hybridization buffer; and
optionally, packaging and instructions for the use of the kit to detect an SNV on an analyte in a test sample.

18. The kit of claim 17, wherein the T2, T3, T4 strands and T1 strand hybridization fragment is biotinylated and attached to a streptavidin-coated substrate or beads.

19. The kit of claim 17, further comprising a plurality of oligonucleotide sets of T2-T2 arm, T3-R strand, T4-T4 arm, and P strand wherein the analyte binding regions of the R strand, P strand, T2 arm, and T4 arm are independently selected for specific bindings to various SNVs on various analytes.

20. A method of identifying a single nucleotide variation in an analyte using the UMB-based hybridization sensor system of claim 1, comprising the steps of:
(i) combining the oligonucleotides of T1, T2-T2 arm, T3-R strand, T4-T4 arm, the UMB probe, the R strand, the P strand and an analyte in hybridization buffer, wherein the P strand is added after mixing the other components;
(ii) subjecting the mixture of (i) to conditions to permit annealing;
(iii) illuminating the second mixture at an excitation wavelength suitable for inducing fluorescence emission by the fluorophore; and
(iv) detecting the fluorescence emitted by the fluorophore, thereby detecting the presence of a single nucleotide variation in the analyte in the test sample.


SEQUENCE LISTING (53 sequences)

SEQ ID NO: 1 - DNA, 24 bp, synthetic construct
  misc_feature 1..24: note = Synthetic
  modified_base 1: mod_base = OTHER; note = fluorescein
  modified_base 24: mod_base = OTHER; note = black hole quencher 1
  source 1..24: mol_type = other DNA
cgcgttcccatacaaccaatcgcg

SEQ ID NO: 2 - DNA, 52 bp, synthetic construct
  misc_feature 1..52: note = Synthetic
  source 1..52: mol_type = other DNA
gtatcagtcattaccagtagtcggacctaggctctcggtctagccacttaac

SEQ ID NO: 3 - DNA, 40 bp, synthetic construct
  misc_feature 1..40: note = Synthetic
  source 1..40: mol_type = other DNA
actactggtaatgactgatactttcggcgcatgggacgtg

SEQ ID NO: 4 - DNA, 30 bp, synthetic construct
  misc_feature 1..30: note = Synthetic
  source 1..30: mol_type = other DNA
cctaggtccggaacgtgaaggtacttatgg

SEQ ID NO: 5 - DNA, 33 bp, synthetic construct
  misc_feature 1..33: note = Synthetic
  source 1..33: mol_type = other DNA
cctaggtccgtttgaacgtgaaggtacttatgg

SEQ ID NO: 6 - DNA, 10 bp, synthetic construct
  misc_feature 1..10: note = Synthetic
  source 1..10: mol_type = other DNA
cctaggtccg

SEQ ID NO: 7 - DNA, 40 bp, synthetic construct
  misc_feature 1..40: note = Synthetic
  source 1..40: mol_type = other DNA
gcctcccgggacgtgttttgttaagtggctagaccgagag

SEQ ID NO: 8 - DNA, 74 bp, synthetic construct
  misc_feature 1..74: note = Synthetic
  source 1..74: mol_type = other DNA
cgcctcccgggacgtgttttgttaagtggctagaccgagagcctaggtccgtttgaacgtgaaggtacttatgg

SEQ ID NO: 9 - DNA, 41 bp, synthetic construct
  misc_feature 1..41: note = Synthetic
  source 1..41: mol_type = other DNA
cgcctcccgggacgtgttttgttaagtggctagaccgagag

SEQ ID NO: 10 - DNA, 39 bp, synthetic construct
  misc_feature 1..39: note = Synthetic
  source 1..39: mol_type = other DNA
cctcccgggacgtgttttgttaagtggctagaccgagag

SEQ ID NO: 11 - DNA, 40 bp, synthetic construct
  misc_feature 1..40: note = Synthetic
  source 1..40: mol_type = other DNA
actactggtaatgactgatactttgttcaagaaattcaac

SEQ ID NO: 12 - DNA, 33 bp, synthetic construct
  misc_feature 1..33: note = Synthetic
  source 1..33: mol_type = other DNA
cctaggtccgtttgaacgtccaggcagctatgg

SEQ ID NO: 13 - DNA, 40 bp, synthetic construct
  misc_feature 1..40: note = Synthetic
  source 1..40: mol_type = other DNA
acttctcctgctagaatttgttaagtggctagaccgagag

SEQ ID NO: 14 - DNA, 41 bp, synthetic construct
  misc_feature 1..41: note = Synthetic
  source 1..41: mol_type = other DNA
aacttctcctgctagaatttgttaagtggctagaccgagag

SEQ ID NO: 15 - DNA, 18 bp, synthetic construct
  misc_feature 1..18: note = Synthetic
  source 1..18: mol_type = other DNA
gttgcacactgccgattg

SEQ ID NO: 16 - DNA, 17 bp, synthetic construct
  misc_feature 1..17: note = Synthetic
  source 1..17: mol_type = other DNA
gttgcacactgccattg

SEQ ID NO: 17 - DNA, 17 bp, synthetic construct
  misc_feature 1..17: note = Synthetic
  source 1..17: mol_type = other DNA
gttgcacactgcgattg

SEQ ID NO: 18 - DNA, 18 bp, synthetic construct
  misc_feature 1..18: note = Synthetic
  source 1..18: mol_type = other DNA
ggttgcacactgccgatt

SEQ ID NO: 19 - DNA, 19 bp, synthetic construct
  misc_feature 1..19: note = Synthetic
  source 1..19: mol_type = other DNA
ggttgcacactgcccgatt

SEQ ID NO: 20 - DNA, 16 bp, synthetic construct
  misc_feature 1..16: note = Synthetic
  source 1..16: mol_type = other DNA
gttgcacactgcattg

SEQ ID NO: 21 - DNA, 16 bp, synthetic construct
  misc_feature 1..16: note = Synthetic
  source 1..16: mol_type = other DNA
gttgcacactggattg

SEQ ID NO: 22 - DNA, 18 bp, synthetic construct
  misc_feature 1..18: note = Synthetic
  source 1..18: mol_type = other DNA
gttgagtaaacgagattg

SEQ ID NO: 23 - DNA, 17 bp, synthetic construct
  misc_feature 1..17: note = Synthetic
  source 1..17: mol_type = other DNA
gttgagtaaacggattg

SEQ ID NO: 24 - DNA, 60 bp, synthetic construct
  misc_feature 1..60: note = Synthetic
  source 1..60: mol_type = other DNA
caaacacgtcccgggaggcggcagtgtgagtaccttcacacgtcccatgcgccgtgctgt

SEQ ID NO: 25 - DNA, 60 bp, synthetic construct
  misc_feature 1..60: note = Synthetic
  source 1..60: mol_type = other DNA
caaacacgtcccgggaggcggcagcgtgagtaccttcacacgtcccatgcgccgtgctgt

SEQ ID NO: 26 - DNA, 60 bp, synthetic construct
  misc_feature 1..60: note = Synthetic
  source 1..60: mol_type = other DNA
caaacacgtcccgggaggcggcaatgtgagtaccttcacacgtcccatgcgccgtgctgt

SEQ ID NO: 27 - DNA, 60 bp, synthetic construct
  misc_feature 1..60: note = Synthetic
  source 1..60: mol_type = other DNA
caaacacgtcccgggaggcggcggtgtgagtaccttcacacgtcccatgcgccgtgctgt

SEQ ID NO: 28 - DNA, 19 bp, synthetic construct
  misc_feature 1..19: note = Synthetic
  source 1..19: mol_type = other DNA
gcagtgtgagtaccttcac

SEQ ID NO: 29 - DNA, 19 bp, synthetic construct
  misc_feature 1..19: note = Synthetic
  source 1..19: mol_type = other DNA
gcagcgtgagtaccttcac

SEQ ID NO: 30 - DNA, 19 bp, synthetic construct
  misc_feature 1..19: note = Synthetic
  source 1..19: mol_type = other DNA
gcaatgtgagtaccttcac

SEQ ID NO: 31 - DNA, 18 bp, synthetic construct
  misc_feature 1..18: note = Synthetic
  source 1..18: mol_type = other DNA
gcagtgtgagtaccttca

SEQ ID NO: 32 - DNA, 18 bp, synthetic construct
  misc_feature 1..18: note = Synthetic
  source 1..18: mol_type = other DNA
gcagcgtgagtaccttca

SEQ ID NO: 33 - DNA, 18 bp, synthetic construct
  misc_feature 1..18: note = Synthetic
  source 1..18: mol_type = other DNA
gcaatgtgagtaccttca

SEQ ID NO: 34 - DNA, 60 bp, synthetic construct
  misc_feature 1..60: note = Synthetic
  source 1..60: mol_type = other DNA
tgccagccattctagcaggagaagttcgtttactgctgcctggagttgaatttcttgaac

SEQ ID NO: 35 - DNA, 60 bp, synthetic construct
  misc_feature 1..60: note = Synthetic
  source 1..60: mol_type = other DNA
tgccagccattctagcaggagaagttcgcttactgctgcctggagttgaatttcttgaac

SEQ ID NO: 36 - DNA, 60 bp, synthetic construct
  misc_feature 1..60: note = Synthetic
  source 1..60: mol_type = other DNA
tgccagccattctagcaggagaagttcgtgtactgctgcctggagttgaatttcttgaac

SEQ ID NO: 37 - DNA, 18 bp, synthetic construct
  misc_feature 1..18: note = Synthetic
  source 1..18: mol_type = other DNA
acttccatgagtgtgacg

SEQ ID NO: 38 - DNA, 17 bp, synthetic construct
  misc_feature 1..17: note = Synthetic
  source 1..17: mol_type = other DNA
gttgcacgctgcgattg

SEQ ID NO: 39 - DNA, 17 bp, synthetic construct
  misc_feature 1..17: note = Synthetic
  source 1..17: mol_type = other DNA
gttgcacattgcgattg

SEQ ID NO: 40 - DNA, 24 bp, synthetic construct
  misc_feature 1..24: note = Synthetic
  source 1..24: mol_type = other DNA
gcagtgtggcagtgtggcagtgtg

SEQ ID NO: 41 - DNA, 24 bp, synthetic construct
  misc_feature 1..24: note = Synthetic
  source 1..24: mol_type = other DNA
gcagcgtggcagcgtggcagcgtg

SEQ ID NO: 42 - DNA, 24 bp, synthetic construct
  misc_feature 1..24: note = Synthetic
  source 1..24: mol_type = other DNA
gcaatgtggcaatgtggcaatgtg

SEQ ID NO: 43 - DNA, 24 bp, synthetic construct
  misc_feature 1..24: note = Synthetic
  source 1..24: mol_type = other DNA
gcggtgtggcggtgtggcggtgtg

SEQ ID NO: 44 - DNA, 20 bp, synthetic construct
  misc_feature 1..20: note = Synthetic
  source 1..20: mol_type = other DNA
gaacgtgaaggtacttatgg

SEQ ID NO: 45 - DNA, 25 bp, synthetic construct
  misc_feature 1..25: note = Synthetic
  source 1..25: mol_type = other DNA
cctaggtccggaacggtgaaggtac

SEQ ID NO: 46 - DNA, 28 bp, synthetic construct
  misc_feature 1..28: note = Synthetic
  source 1..28: mol_type = other DNA
cctaggtccgtttgaacggtgaaggtac

SEQ ID NO: 47 - DNA, 10 bp, synthetic construct
  misc_feature 1..10: note = Synthetic
  source 1..10: mol_type = other DNA
cctaggtccg

SEQ ID NO: 48 - DNA, 17 bp, synthetic construct
  misc_feature 1..17: note = Synthetic
  source 1..17: mol_type = other DNA
cgcctcccgggacgtgt

SEQ ID NO: 49 - DNA, 21 bp, synthetic construct
  misc_feature 1..21: note = Synthetic
  source 1..21: mol_type = other DNA
actactggtaatgactgatac

SEQ ID NO: 50 - DNA, 10 bp, synthetic construct
  misc_feature 1..10: note = Synthetic
  source 1..10: mol_type = other DNA
cctaggtccg

SEQ ID NO: 51 - DNA, 21 bp, synthetic construct
  misc_feature 1..21: note = Synthetic
  source 1..21: mol_type = other DNA
gttaagtggctagaccgagag

SEQ ID NO: 52 - DNA, 20 bp, synthetic construct
  source 1..20: mol_type = other DNA
gaacgtgaaggtacttatgg

SEQ ID NO: 53 - DNA, 15 bp, synthetic construct
  source 1..15: mol_type = other DNA
gaacggtgaaggtac